(19) United States
(12) Patent Application Publication
(10) Pub. No.: US 2019/0231436 A1
(43) Pub. Date: Aug. 1, 2019

(54) ANATOMICAL MODEL FOR POSITION PLANNING AND TOOL GUIDANCE OF A MEDICAL TOOL

(71) Applicant: KONINKLIJKE PHILIPS N.V., EINDHOVEN (NL)

(72) Inventors: Ashish PANSE, BURLINGTON, MA (US); Molly Lara FLEXMAN, MELROSE, MA (US); Aleksandra POPOVIC, BOSTON, MA (US)

(73) Assignee: KONINKLIJKE PHILIPS N.V., EINDHOVEN (NL)

(21) Appl. No.: 16/336,603

(22) PCT Filed: Sep. 28, 2017
(86) PCT No.: PCT/EP2017/074582

§ 371 Date: Mar. 26, 2019

Related U.S. Application Data
(60) Provisional application No. 62/402,263, filed on Sep. 30, 2016.
(60) Provisional application No. 62/447,051, filed on Jan. 17, 2017.

(51) Int. Cl. A61B 34/10; G16H 50/50; A61B 5/06; A61B 5/00; A61B 34/20; A61B 34/30; A61B 6/03

(52) U.S. Cl. CPC A61B 34/10; G16H 50/50; A61B 5/061; A61B 5/745; A61B 2034/107; A61B 34/30; A61B 6/032; A61B 2034/104; A61B 2034/105; A61B 34/20

(57) ABSTRACT

An anatomical model medical suite (10) for executing an anatomical model medical procedure including an anatomical model (40) physically representative of the patient anatomy. The anatomical model (40) medical suite (10) employs a medical procedure controller (90) for controlling a position planning and/or a tool guidance of the medical tool (20) relative to the patient anatomy as derived from a position planning and/or a tool guidance of the medical tool (20) relative to the anatomical model (40) and/or a tool replica (30) relative to the anatomical model (40). The medical tool (20) is for conducting an imaging, a diagnosis and/or a treatment of the patient anatomy. The tool replica (30) is a physical representation of the medical tool (20). The anatomical model medical suite (10) may further employ an image system (50), a tracking system (60), robotic system (70) and/or an augmented reality system (300).

### FIELD OF THE INVENTION

The present disclosure generally relates to various medical procedures (e.g., laparoscopic surgery, neurosurgery, spinal surgery, natural orifice transluminal surgery, cardiology, pulmonary/bronchoscopy surgery, biopsy, ablation, and diagnostic interventions). The present disclosure specifically relates to an anatomical model for position planning and tool guidance during a medical procedure.

### BACKGROUND OF THE INVENTION

Traditional surgery relies on individual skills of surgeons, particularly dexterity of a surgeon is limited to the surgeon's hands and rigid instruments. This issue is particularly amplified in minimally invasive surgery or natural orifice surgery where space to operate is limited by the entry point and the anatomy. To address this issue, surgical robots are designed to improve the surgeon's dexterity inside the body. Such surgical robots may be in the form of multi-arm systems, flexible robots and catheter robots.

The robotic systems are controlled by the surgeon using different input mechanisms that may include joysticks, haptic interfaces, head-mounted displays, computer interfaces (e.g., a keyboard, a mouse, etc.). As the surgeon controls the robotic system, visual feedback of the operating site is provided by endoscopic cameras or rendered presentation of images from other imaging modalities (e.g. CT, MRI, X-ray, and ultrasound).

More particularly, in order to improve the surgeon's dexterity, surgical robots may usually have six (6) or more degrees of freedom making them unintuitive to control. This issue is amplified in constraint spaces, such as minimally invasive surgery or natural orifice surgery, and hyper-redundant robots, such as snake robots. Control of these robots is usually performed using handles that are complex to operate and are usually associated with a steep learning curve. In addition, in minimally invasive procedures, surgeons may have a very limited visual feedback of the device and anatomy. For example, in cardiac interventions, a Transesophageal Echocardiography (TEE) probe and X-ray images are used to generate real-time images of heart and valves. In oncology surgery, the images are provided by an endoscope. These images may be difficult to interpret and relate to anatomy. This potential problem is amplified by the fact that the images are displayed on two-dimensional (“2D”) screens and interaction (i.e., rotation, translation) with the models, which is necessary to obtain full three-dimensional (“3D”) information, disrupts the workflow and adds to the procedure time.

Additionally, 3D printing is growing in popularity for many applications. In the medical space, a doctor may use a 3D printed anatomical model of a specific patient anatomy to visualize medical procedure(s) involving the patient anatomy for purposes of facilitating a mental planning of the medical procedure(s). For example, a 3D printed anatomical model of an aortic valve has been used to visualize a deployment of a trans-catheter valve within the 3D printed anatomical model of the aortic valve to thereby facilitate a mentally planning by the doctor on the appropriate actions for sizing, positioning, and successfully deploying the trans-catheter valve.

Furthermore, it may be challenging at time to interact with 3D images, models, and data via a mouse, a keyboard and a 2D display. Augmented reality may be used to help with this problem by providing new ways to visualize 3D information and to allow users to that interact directly with 3D images, models, and data.

More particularly, augmented reality generally refers to when a live image stream is supplemented with additional computer-generated information. The live image stream may be visualized via an operator eye, cameras, smart phones, tables, etc. This image stream is augmented via display to the operator that may be accomplished via glasses, contact lenses, projections or on the live image stream device itself (e.g., a smart phone, a tablet, etc.). Also, in complex anatomies, it is often difficult to get the best view during image guided interventions, particularly in view of the fact that most imaging systems cannot reach every possible position (i.e., location and orientation) and the positions that are available are not always intuitive to the operator. For example, a robotic intensity modulated radiation therapy (“IMRT”) machine (e.g., CyberKnife® System) may a constrained robotic manipulator with a lightweight linear accelerator. By further example, a robotic C-arm (e.g., Siemens Artis Zeego) may be used for a diagnostic 2D and 3D x-ray imaging. Such systems are maneuvered within workspace constraints, which is achieved by a combination of software and hardware implementation.

### SUMMARY OF THE INVENTION

The present disclosure describes improvements to medical procedures and medical suites for intuitive control of medical tools during medical procedures by a novel and unique incorporation of an anatomical model as a physical representation of patient anatomy and an optional incorporation of a tool replica as a physical representation of a medical tool. Any such physical representation is registered to both the patient anatomy and/or a corresponding medical tool whereby the physical representation may be utilized to guide a medical procedure (e.g., minimally invasive therapy) to thereby giving a user some experience and benefits, if not all, of an open procedure.

More particularly, an anatomical model of a patient anatomy (e.g., a 3D printed anatomical model, a standard atlas anatomical model or a hologram of the patient anatomy) may be utilized for position planning and/or tool guidance, pre-operative or intra-operative, of medical tool(s) relative to the patient anatomy. Furthermore, physiologically information, planning information and/or guidance feedback information may be incorporated into and/or related to the anatomical model.

The present disclosure further describes improvements to medical procedures and medical suites involving an incorporation of augmented reality to provide new ways to visualize and directly interact with 3D models, images and data.

The present disclosure additionally describes improvements to medical procedures and medical suites for facilitating a positioning of an imaging system relative to a patient in order to obtain the best possible views of an anatomy of interest during an image guided intervention within the constraints of achievable positions of the imaging system.

For purposes of describing and claiming the inventions of the present disclosure:

(1) the term “medical procedure” broadly encompasses all diagnostic, surgical and interventional procedures, as known in the art of the present disclosure or hereinafter conceived, for an imaging, a diagnosis and/or a treatment of a patient anatomy;

(2) the term “medical suite” broadly encompasses all medical suites, as known in the art of the present disclosure and hereinafter conceived, incorporating systems and medical tools necessary for the performance of one or more specific types of medical procedures. Examples of such suites include, but are not limited to, the Allure Xper Interventional Suites. Examples of such systems include, but are not limited to, imaging systems, tracking systems, robotic systems and augmented reality systems;

(3) the term “imaging system” broadly encompasses all imaging systems, as known in the art of the present disclosure and hereinafter conceived, for imaging a patient anatomy. Examples of an imaging system include, but is not limited to, a standalone x-ray imaging system, a mobile x-ray imaging system, an ultrasound imaging system (e.g., TEE, TTE, IVUS, ICE), computed tomography (“CT”) imaging system, positron emission tomography (“PET”) imaging system, and magnetic resonance imaging (“MRI”) system;

(4) the term “tracking system” broadly encompasses all tracking systems, as known in the art of the present disclosure and hereinafter conceived, for tracking objects within a coordinate space. Examples of a tracking system include, but is not limited to, an electromagnetic (“EM”) tracking system (e.g., the Auora® electromagnetic tracking system), an optical-fiber based tracking system (e.g., Fiber-Optic RealShape (“FORS”) tracking system), an ultrasound tracking system (e.g., an InSitu or image-based US tracking system), an optical tracking system (e.g., a Polaris optical tracking system), a radio frequency identification tracking system and a magnetic tracking system;

(5) the term “FORS sensor” broadly encompasses an optical fiber structurally configured as known in the art for extracting high density strain measurements of the optical fiber derived from light emitted into and propagated through the optical fiber and reflected back within the optical fiber in an opposite direction of the propagated light and/or transmitted from the optical fiber in a direction of the propagated light. An example of a FORS sensor includes, but is not limited to, an optical fiber structurally configured under the principle of Optical Frequency Domain Reflectometry (OFDR) for extracting high density strain measurements of the optical fiber derived from light emitted into and propagated through the optical fiber and reflected back within the optical fiber in an opposite direction of the propagated light and/or transmitted from the optical fiber in a direction of the propagated light via controlled grating patterns within the optical fiber (e.g., Fiber Bragg Gratings), a characteristic backscatter of the optical fiber (e.g., Rayleigh backscatter) or any other arrangement of reflective element(s) and/or transmissive element(s) embedded, etched, imprinted, or otherwise formed in the optical fiber. Commercially and academically, Fiber-Optic RealShape may also be known as optical shape sensing (“OSS”); and

(6) the term “robotic system” broadly encompasses all robotic systems, as known in the art of the present disclosure and hereinafter conceived, for robotically guiding a medical tool within a coordinate space. Examples of a robotic system include, but is not limited to, the da Vinci® Robotic System, the Medrobotics Flex® Robotic System, the Magellan™ Robotic System, and the CorePath® Robotic System;

(7) the term “augmented reality system” broadly encompasses all augmented reality systems, as known in the art of the present disclosure and hereinafter conceived, for a physical interaction with hologram. Examples of an augmented reality systems include, but is not limited to, augmented reality systems commercially available from Google, Microsoft, Meta, Magic Leap and Vusix;

(8) the term “medical tool” broadly encompasses, as understood in the art of the present disclosure and hereinafter conceived, a tool, an instrument, a device or the like for conducting an imaging, a diagnosis and/or a treatment of a patient anatomy. Examples of a medical tool include, but are not limited to, guidewires, catheters, scalpels, cauterizers, ablation devices, balloons, stents, endografts, atherectomy devices, clips, needles, forceps, k-wires and associated drivers, endoscopes, ultrasound probes, X-ray devices, awls, screwdrivers, osteotomes, chisels, mallets, curettes, clamps, forceps, periosteomes and j-needles;

(9) the term “position planning” broadly encompasses, as understood in the art of the present disclosure and exemplary described herein, an operation of a system or a device in planning a positioning of a medical tool relative to a patient anatomy for a purpose of conducting an imaging, a diagnosis and/or a treatment of the patient anatomy. A non-limiting example of such systems and devices is a controller housed within or linked to a workstation whereby the controller provides a graphical user interface for selectively editing an image of the patient anatomy (e.g., slicing, cropping and/or rotating the image) to thereby illustrate a planned positioning of the medical tool relative to the patient anatomy (e.g., a delineation of a target for a distal end/operating piece of the medical tool that is spaced from or on the patient anatomy, or a delineation of a path of the distal end/operating piece spatially and/or contiguously traversing an exterior and/or an interior of the patient anatomy);

(10) the term “tool guidance” broadly encompasses, as understood in the art of the present disclosure and exemplary described herein, an operation of a system or a device in controlling a positioning of a medical tool relative to a patient anatomy for a purpose of conducting an imaging, a diagnosis and/or a treatment of the patient anatomy. A non-limiting example of such systems and devices is a controller of a workstation whereby the controller provides a user input device (e.g., a joystick) for translationally, rotationally and/or pivotally a steerable medical tool relative to the patient anatomy, particularly in accordance with a position planning as illustrated in a tracked imaging of the medical tool relative to the patient anatomy. A further non-limiting example is a robotic system for controlling a translation, a rotation and/or a pivoting of a robotic actuated medical tool relative to the patient anatomy, particularly in accordance with an execution by a controller of the robotic system of planning data informative of the position planning;

(11) the term “anatomical model medical procedure” broadly encompasses a medical procedure incorporating the inventive principles of the present disclosure for a position planning and/or a tool guidance of a medical tool based on an anatomical model of a patient anatomy as exemplary described herein;

(12) the term “anatomical model medical suite” broadly encompasses a medical suite incorporating inventive principles of the present disclosure for a position planning and/or a tool guidance of a medical tool based on an anatomical model of a patient anatomy as exemplary described herein; and

(13) the term “anatomical model” broadly encompasses any type of physical representation of a patient anatomy suitable for a position planning and/or a tool guidance of a medical tool relative to the patient anatomy including, but not limited to, 3D printed anatomical model, a standard atlas anatomical model and a holographic anatomical model as exemplary described herein. The anatomical model may be patient-specific, such as, for example, via a manufacturing of the anatomical model from an imaging of the patient anatomy, or a delineation of the anatomical model from the imaging of the patient anatomy for facilitating a selection or a morphing of a generic anatomical model, particularly manufactured from an anatomical atlas, or a holographic anatomical model generated from an imaging of the patient anatomy. Alternatively, the anatomical model may be non-patient-specific, such as, for example, a generic anatomical model manufactured/selected from an anatomical atlas, or a holographic anatomical model generated from a generic anatomical model selected from an anatomical atlas, or any type of object physically representative of the patient anatomy;

(14) the term “tool replica” broadly encompasses any type of physical representation of a medical tool that is structurally equivalent or functionally equivalent to a physical operation of the medical tool as exemplary described herein. Examples of a tool replica include, but are not limited to, a model of a medical tool, a robot, a laser pointer, an optical projector, a scaled down model of an imaging system and holographic tools generated by interactive tools of an augmented reality system;

(15) the term “controller” broadly encompasses all structural configurations, as understood in the art of the present disclosure and as exemplary described herein, of an application specific main board or an application specific integrated circuit for controlling an application of various inventive principles of the present disclosure as subsequently described herein. The structural configuration of the controller may include, but is not limited to, processor(s), computer-usable/computer readable storage medium(s), an operating system, application module(s), peripheral device controller(s), slot(s) and port(s). A controller may be housed within or linked to a workstation. Examples of a “workstation” include, but are not limited to, an assembly of one or more computing devices, a display/monitor, and one or more input devices (e.g., a keyboard, joysticks and mouse) in the form of a standalone computing system, a client computer of a server system, a desktop or a tablet.

(16) the descriptive labels for term “controller” herein facilitates a distinction between controllers as described and claimed herein without specifying or implying any additional limitation to the term “controller”;

(17) the term “module” broadly encompasses a module incorporated within or accessible by a controller consisting of an electronic circuit and/or an executable program (e.g., executable software stored on non-transitory computer readable medium(s) and/or firmware) for executing a specific application;

(18) the descriptive labels for term “module” herein facilitates a distinction between modules as described and claimed herein without specifying or implying any additional limitation to the term “module”;

(19) the terms “data” and “command” broadly encompasses all forms of a detectable physical quantity or impulse (e.g., voltage, current, or magnetic field strength) as understood in the art of the present disclosure and as exemplary described herein for transmitting information and/or instructions in support of applying various inventive principles of the present disclosure as subsequently described herein. Data/command communication between components of an anatomical model medical suite of the present disclosure may involve any communication method as known in the art of the present disclosure including, but not limited to, data/command transmission/reception over any type of wired or wireless datalink and a reading of data/commands uploaded to a computer-usable/computer readable storage medium; and

(20) the descriptive labels for term “data” herein facilitates a distinction between data as described and claimed herein without specifying or implying any additional limitation to the term “data”.

A first embodiment of the inventions of the present disclosure is an anatomical model medical suite for executing an anatomical model medical procedure including an anatomical model physically representative of the patient anatomy (e.g., a 3D printed anatomical model, a standard atlas anatomical model or a holographic anatomical model, all of which may be patient-specific or non-patient-specific).

The anatomical model medical suite employs a medical tool for conducting an imaging, a diagnosis and/or a treatment of the patient anatomy.

The anatomical model medical suite further employs a medical procedure controller for controlling a position planning and/or a tool guidance of the medical tool relative to the patient anatomy derived from a position planning and/or a tool guidance of the medical tool relative to the anatomical world and/or a tool replica relative to the anatomical model.

The tool replica physically represents the medical tool.

A second embodiment of the inventions of the present disclosure is an anatomical model medical suite for executing an anatomical model medical procedure including a medical tool for conducting an imaging, a diagnosis and/or a treatment of the patient anatomy, and further including an anatomical model physically representative of the patient anatomy (e.g., a 3D printed anatomical model, a standard atlas anatomical model or a holographic anatomical model, all of which may be patient-specific or non-patient-specific).

The anatomical model medical suite employs a medical procedure controller and further employs an imaging system, a tracking system, a robotic system and/or an augmented reality system operating in conjunction with the medical procedure controller during a pre-operative phase and/or an intra-operative phase of the imaging, the diagnosis and/or the treatment of the patient anatomy.

The medical procedure controller controls a position planning and/or a tool guidance of the medical tool relative to the patient anatomy derived from a position planning and/or a tool guidance of the medical tool relative to the anatomical model and/or a tool replica relative to the anatomical model.

For example as exemplary described herein, the anatomical model medical procedure may pre-operatively involve a manual tool guidance or a robotic tool guidance of the tool replica relative to the anatomical model of the patient anatomy for generating plan data informative of a position planning of the medical tool relative to the patient anatomy, and may intra-operatively involve a manual tool guidance or a robotic tool guidance of the medical tool relative to the patient anatomy in accordance with the plan data.

Additionally, physiologically information may be incorporated into and/or related to the anatomical model to enhance the plan planning and/or tool guidance activities.

More particularly for a Cox-Maze procedure, pre-operatively, an optical beam of a laser pointer as tracked by the tracking system may be manually guided across an exterior of an anatomical model of a patient heart as a simulation of a catheter ablation of the patient heart whereby the medical procedure controller controls a generation of plan data informative of the simulated catheter ablation of the patient heart. Intra-operatively, the medical procedure controller controls a robotic tool guidance by the robotic system of an ablation catheter across the patient heart in accordance with the plan data to perform the simulated catheter ablation.

Additionally, the anatomical model may be color-coded or texture-coded to identify safe/operable regions and unsafe/inoperable regions of the patient heart for the Cox-Maze procedure whereby the simulated catheter ablation may avoid the unsafe/inoperable regions.

Alternatively, pre-operatively, the optical beam of the laser pointer may be robotically guided by the robotic system across the exterior of the patient heart as the simulation of a catheter ablation of the patient heart whereby the medical procedure controller controls a generation of plan data informative of the simulated catheter ablation of the patient heart.

By further example as exemplary described herein, the anatomical model medical procedure may pre-operatively involve planning information incorporated within the anatomical model of the patient anatomy whereby the planning information is illustrative of a planned path of a medical tool relative to the patient anatomy, and may intra-operatively involve a robotic tool guidance of the medical tool relative to the patient anatomy as a tool replica is manually guided relative to the planned path incorporated within the anatomical model of the patient anatomy.

More particularly for a knee-replacement procedure, pre-operatively, the medical procedure controller controls a position planning of surgical paths across the patient knee within an image of the patient knee as imaged by the imaging system whereby the medical procedure controller generates an anatomical model profile for the manufacturing (e.g., a 3D printing) of an anatomical model of the patient knee incorporating the surgical paths. Intra-operatively, medical procedure controller controls a robotic tool guidance of a robotic saw by a robotic system across the patient knee to form the surgical paths in accordance with a manual tool guidance of a tracked replica saw by the tracking system saw across the surgical paths of the anatomical model of the patient knee or in accordance with a robotic tool guidance by an additional robotic system of the saw across the surgical paths of the anatomical model of the patient knee.

By further example as exemplary described herein, the anatomical model medical procedure may pre-operatively involve a manufacture and/or a coating of an anatomical model of a patient anatomy from material susceptible to a color change in response to an application of a heat or a light to the material, and may intra-operatively involve a robotic tool guidance by a robotic system of a laser pointer relative to the anatomical model of the patient anatomy that mimics a manual tool guidance of a medical tool relative to the patient anatomy whereby heat/light applied by the laser pointer on the anatomical model of the patient anatomy illustrates the manual tool guidance of the medical tool relative to the patient anatomy.

More particularly for a Cox-Maze procedure, pre-operatively, an anatomical model of a patient heart is manufactured or coated from material susceptible to a color change in response to an application of a heat or a light to the material. Intra-operatively, the medical procedure controller controls a robotic tool guidance by a robotic system of a laser pointer relative to the anatomical model of the patient heart that mimics a manual tool guidance of a medical tool relative to the patient heart whereby heat/light applied by the laser pointer on the anatomical model of the patient heart illustrates the manual tool guidance of the ablation catheter across the patient heart.

By further example as exemplary described herein, the anatomical model medical procedure may pre-operatively involve a manual or robotic manipulation of an encoded plane selector with respect to an anatomical model of the patient anatomy. The position of the plane selector is used to extract a particular slice from a preoperative 3D image (e.g., ultrasound, MRI, CT, etc.) of the patient anatomy. Alternatively, the plane selector position may be used to intra-operatively control a positioning of an imaging device (e.g., control of an angulation of an interventional x-ray c-arm, of a positioning of a robotically controlled TEE probe, or of a focal depth/field-of-view of an ultrasound transducer).

By further example as exemplary described herein, the anatomical model may pre-operatively involve a generation of a holographic anatomical model from an image of the patient anatomy or a generic standard anatomical model whereby use interaction with the holographic anatomical model serves as a basis for a path planning and/or a tool guidance. More particularly, a desired view of the patient anatomy may be planned and/or guided via a user interaction with the holographic anatomical model, pre-operatively or intra-operatively, whereby an intra-operative imaging system may be operated to achieve the desired view of the patient anatomy. Such interaction with the holographic anatomical model may be performed within kinematic constraints of the intra-operative imaging system.

The foregoing embodiments and other embodiments of the inventions of the present disclosure as well as various features and advantages of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of present disclosure being defined by the appended claims and equivalents thereof.

### BRIEF DESCRIPTION OF THE DRAWINGS

FIG. 1A illustrates a block diagram of a first exemplary embodiment of an anatomical model medical suite in accordance with the inventive principles of the present disclosure.

FIG. 1B illustrates a block diagram of a first exemplary embodiment of an anatomical model medical procedure in accordance with the inventive principles of the present disclosure.

FIG. 2A illustrates a block diagram of an exemplary embodiment of an anatomical model medical workstation for executing an image based model manufacture method in accordance with the inventive principles of the present disclosure.

FIG. 2B illustrates a block diagram of an exemplary embodiment of an anatomical model medical workstation for executing an image based model selection method in accordance with the inventive principles of the present disclosure.

FIG. 3A illustrates a work flow of an exemplary embodiment of an image based model manufacture method in accordance with the inventive principles of the present disclosure.

FIG. 3B illustrates a work flow of an exemplary embodiment of an image based model selection method in accordance with the inventive principles of the present disclosure.

FIGS. 4A-4F illustrates exemplary embodiments of anatomical model enhancements in accordance with the inventive principles of the present disclosure.

FIG. 5A illustrates a block diagram of an exemplary embodiment of an anatomical model medical workstation for executing a non-model based position planning method in accordance with the inventive principles of the present disclosure.

FIG. 5B illustrates a block diagram of an exemplary embodiment of an anatomical model medical workstation for executing an anatomical model based pre-operative/intra-operative based position planning method in accordance with the inventive principles of the present disclosure.

FIGS. 6A and 6B illustrate work flows of exemplary embodiments of a non-model based position planning incorporated within an anatomical model in accordance with the inventive principles of the present disclosure.

FIGS. 6C and 6D illustrate work flows of exemplary embodiments of an anatomical model based pre-operative/intra-operative position planning incorporated within an anatomical model in accordance with the inventive principles of the present disclosure.

FIG. 7A illustrates a block diagram of an exemplary embodiment of an anatomical model medical workstation for executing a non-model based tool guidance method in accordance with the inventive principles of the present disclosure.

FIG. 7B illustrates a block diagram of an exemplary embodiment of an anatomical model medical workstation for executing an anatomical model based pre-operative tool guidance method in accordance with the inventive principles of the present disclosure.

FIG. 7C illustrates a block diagram of an exemplary embodiment of an anatomical model medical workstation for executing an anatomical model based intra-operative tool guidance method in accordance with the inventive principles of the present disclosure.

FIG. 8A illustrates a work flow of an exemplary embodiment of non-model based tool guidance method in accordance with the inventive principles of the present disclosure.

FIG. 8B illustrates a work flow of an exemplary embodiment of an anatomical model based pre-operative tool guidance method in accordance with the inventive principles of the present disclosure.

FIG. 8C illustrates a work flows of exemplary embodiments of an anatomical model based intra-operative tool guidance method in accordance with the inventive principles of the present disclosure.

FIG. 9A illustrates a block diagram of a second exemplary embodiment of an anatomical model medical suite in accordance with the inventive principles of the present disclosure.

FIG. 9B illustrates a block diagram of a second exemplary embodiment of an anatomical model medical procedure in accordance with the inventive principles of the present disclosure.

FIG. 10A illustrates a first exemplary embodiment of a three-dimensional holographic anatomical model of an anatomical model in accordance with the inventive principles of the present disclosure.

FIG. 10B illustrates a second exemplary embodiment of a three-dimensional holographic anatomical model of an anatomical model in accordance with the inventive principles of the present disclosure.

FIG. 11A illustrates a first exemplary embodiment of a user interaction with the holographic anatomical model shown in FIG. 10A in accordance with the inventive principles of the present disclosure.

FIG. 11B illustrates a second exemplary embodiment of a user interaction with the holographic anatomical model shown in FIG. 10A in accordance with the inventive principles of the present disclosure.

FIG. 11C illustrates a third exemplary embodiment of a user interaction with the holographic anatomical model shown in FIG. 10A in accordance with the inventive principles of the present disclosure.

FIG. 12 illustrates an exemplary embodiment of a flowchart representative of a kinematic control method in accordance with the inventive principles of the present disclosure.

FIG. 13 illustrates an exemplary schematic diagram of a third embodiment of an anatomical model medical suite in accordance with the inventive principles of the present disclosure.

### DETAILED DESCRIPTION OF THE PREFERRED EMBODIMENTS

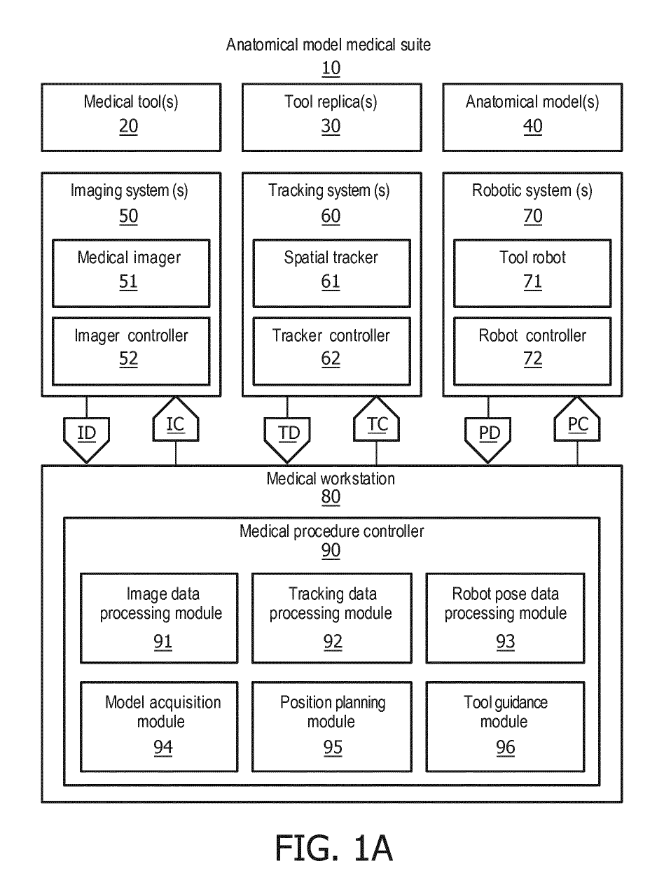

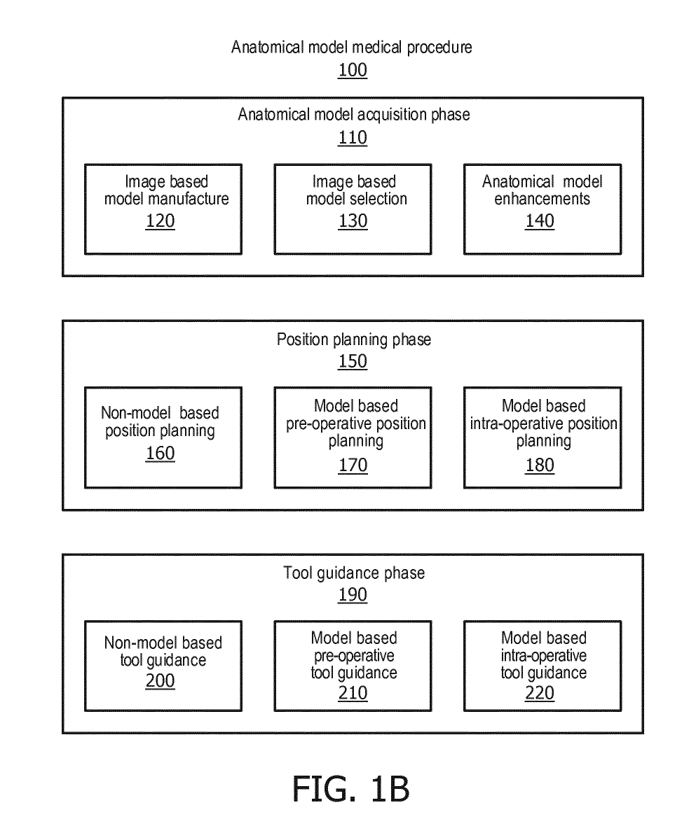

To facilitate an understanding of the present disclosure, the following description of FIG. 1A teaches basic inventive principles of an exemplary anatomical model medical suite of the present disclosure. From this description, those having ordinary skill in the art will appreciate how to apply the inventive principles of the present disclosure to making and using numerous and varied embodiments of anatomical model medical suites of the present disclosure.

Referring to FIG. 1A, an anatomical model medical suite 10 of the present disclosure employs one or more medical tools 20, one or more optional tool replicas 30 and one or more anatomical models 40.

Medical tools 20 are utilized to conduct an imaging, a diagnosis and/or a treatment of a patient anatomy in accordance with a medical procedure as known in the art of the present disclosure. Examples of a medical tool include, but are not limited to, guidewires, catheters, scalpels, cauterizers, ablation devices, balloons, stents, endografts, atherectomy devices, clips, needles, forceps, k-wires and associated drivers, endoscopes, ultrasound probes, X-ray devices, awls, screwdrivers, osteotomes, chisels, mallets, curettes, clamps, forceps, periosteomes and j-needles.

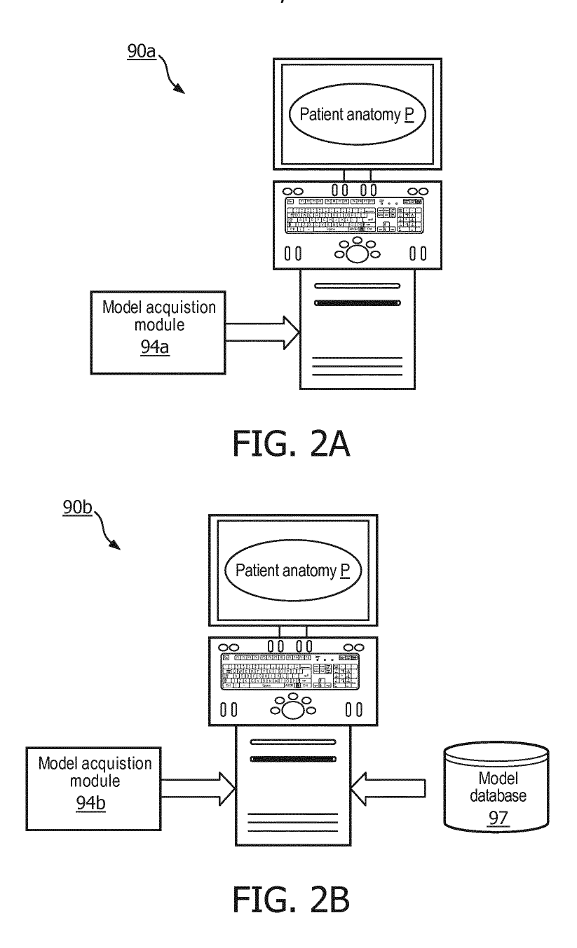

In practice, the specific type(s) of medical tool(s) 20 employed by anatomical model medical suite 10 are dependent upon the specific type(s) of anatomical model medical procedure(s) to be performed via anatomical model medical suite 10. For clarity purposes in describing the inventions of the present disclosure, described embodiments of medical tools 20 for FIGS. 2-8 will be limited to an ablation catheter, a robotic saw and a CT c-arm. Nonetheless, those having ordinary skill in the art of the present disclosure will appreciate the numerous and varied embodiments of a medical tool applicable to the inventions of the present disclosure.

Also in practice, a medical tool 20 may be a standard component of anatomical model medical suite 10 employable for one or more anatomical model medical procedures performed by anatomical model medical suite 10, or a medical tool selectively acquired for a specific anatomical model medical procedures to be performed via anatomical model medical suite 10.

A tool replica 30 is a physical representation of a medical tool 20 that is structurally equivalent or functionally equivalent to a physical operation of the medical tool 20 as exemplary described herein. Examples of a tool replica 30 include, but are not limited to, a model of a medical tool, a model of a robot, a laser pointer and an optical projector;

In practice, the specific type(s) of tool replica(s) 30 employed by anatomical model medical suite 10 are dependent upon the specific type(s) of medical tool(s) 20 employed by anatomical model medical suite 10. For clarity purposes in describing the inventions of the present disclosure, described embodiments of tool replica 30 for FIGS. 2-8 will be limited to a laser point and a robotic saw replica. Nonetheless, those having ordinary skill in the art of the present disclosure will appreciate the numerous and varied embodiments of a tool replica applicable to the inventions of the present disclosure.

Also in practice, a tool replica 30 may be a standard component of anatomical model medical suite 10 employable for one or more anatomical model medical procedures performed by anatomical model medical suite 10, or a tool replica manufactured or selectively acquired for a specific anatomical model medical procedures to be performed via anatomical model medical suite 10.

An anatomical model 40 is physical representation of a patient anatomy that is the subject of the medical procedure as will be further described herein. In practice, the specific type(s) of anatomical model(s) 40 employed by anatomical model medical suite 10 are dependent upon the subject patient anatomy of anatomical model medical procedure(s) to be performed via anatomical model medical suite 10. Also in practice, an anatomical model 40 may be patient-specific via a manufacturing of the anatomical model 40 from an imaging of the patient anatomy or a delineation of the anatomical 40 model from the imaging of the patient anatomy for facilitating a selection or a morphing of a generic anatomical model, particularly manufactured from an anatomical atlas. Alternatively, an anatomical model 40 may be non-patient specific, such as, for example, a generic anatomical model, particularly manufactured from an anatomical atlas, or any type of object physically representative of the patient anatomy.

In practice, a non-patient-specific anatomical model 40 may be a standard component of anatomical model medical suite 10 employable for one or more anatomical model medical procedures performed by anatomical model medical suite 10.

For clarity purposes in describing the inventions of the present disclosure, described embodiments of anatomical model 40 for FIGS. 2-8 will be limited to anatomical models of a patient heart, a patient knee and a patient liver. Nonetheless, those having ordinary skill in the art of the present disclosure will appreciate the numerous and varied embodiments of an anatomical model applicable to the inventions of the present disclosure.

In practice, an anatomical model 40 may partially or entirely physically represent the subject patient anatomy, and the anatomical model 40 may be solid, or partially or entirely hollow.

Still referring to FIG. 1A, anatomical model medical suite 10 of the present disclosure may employ one or more imaging system(s) 50.

In practice, when employed, an imaging system 50 may be a standard component of anatomical model medical suite 10 employable for one or more anatomical model medical procedures performed by anatomical model medical suite 10, or selectively acquired for a specific anatomical model medical procedures to be performed via anatomical model medical suite 10.

Further when employed, an imaging system 50 includes a medical imager 51 for implementing an imaging modality as known in the art of the present disclosure. Examples of imaging modalities implemented by medical imager 51 include, but are not limited to, Computed Tomography (“CT”), Magnetic Resonance Imaging (“MRI”), Positron Emission Tomography (“PET”), ultrasound (“US”), X-ray, and endoscopic.

Each imaging system 50 may further include an imaging controller 52 structurally configured for controlling a generation by a medical imager 51 of imaging data ID illustrative of two-dimensional (“2D”) image(s) and/or a three-dimensional (“3D”) image of a subject patient anatomy, medical tool(s) 20, tool replica(s) 30 and/or anatomical model(s) 40 in accordance with the imaging modality.

In practice, when employed, the specific type(s) of imaging system(s) 50 employed by anatomical model medical suite 10 are selected based upon the specific type(s) of anatomical model medical procedure(s) to be performed via anatomical model medical suite 10.

Also in practice, an imaging system 50 may be utilized during a pre-operative phase and/or an intra-operative phase of anatomical model medical procedure as will be further described herein.

Further in practice, alternative to employing an imaging system 50, anatomical model medical suite 10 may be remote communication with an imaging system 50 for receiving imaging data ID in real-time as generated by the imaging system 50 and/or employ storage (not shown) (e.g., a database) for an uploading/downloading of imaging data ID previously generated by the imaging system 50.

Still referring to FIG. 1A, anatomical model medical suite 10 of the present disclosure may employ one or more tracking system(s) 60.

In practice, when employed, a tracking system 60 may be a standard component of anatomical model medical suite 10 employable for one or more anatomical model medical procedures performed by anatomical model medical suite 10, or a tracking system 60 selectively acquired for a specific anatomical model medical procedures to be performed via anatomical model medical suite 10.

Further when employed, a tracking system 60 includes a spatial tracker 61 for implementing a tracking scheme as known in the art of the present disclosure (e.g., signal/field/optical generators, emitters, transmitters, receivers and/or sensors). Examples of tracking schemes implemented by spatial tracker 61 include, but are not limited to, a Fiber-Optic RealShape (“FORS”) sensor tracking, an electro-magnetic tracking, an optical tracking with cameras, a camera image-based tracking, and mechanical digitization tracking.

Each tracking system 60 may further include a tracking controller 62 structurally configured for controlling a generation by spatial tracker 61 of tracking data TD informative of a tracking of a subject patient anatomy, medical tool(s) 20, tool replica(s) 30 and/or anatomical model(s) 40 within one or more coordinate spaces in accordance with the tracking scheme.

In practice, when employed, the specific type(s) of tracking system(s) 60 employed by anatomical model medical suite 10 are selected based upon the specific type(s) of subject patient anatomy, medical tool(s) 20, tool replica(s) 30 and/or anatomical model(s) 40 within the coordinate space(s).

Also in practice, a tracking system 60 may be utilized during a pre-operative phase and/or an intra-operative phase of an anatomical model medical procedure as will be further described herein.

Still referring to FIG. 1A, anatomical model medical suite 10 of the present disclosure may employ one or more robotic system(s) 70.

In practice, when employed, a robotic system 70 may be a standard component of anatomical model medical suite 10 employable for one or more anatomical model medical procedures performed by anatomical model medical suite 10, or a robotic system 70 selectively acquired for a specific anatomical model medical procedures to be performed via anatomical model medical suite 10.

Further when employed, a robotic system 70 includes a tool robot 71 for guiding a medical tool 20 or a tool replica 30 along a path relative to a subject patient anatomy or an anatomical model 40. Examples of tool robot 71 include, but are not limited to:
(1) a rigid robot having one or more joints and a plurality of links (e.g., a six degree of freedom robot or a remote center of motion robot);
(2) a snake robot having a plurality of joints actuatable via geared motor coupling or tendon driven;
(3) a robot for supporting catheters and similar medical tools (e.g., a robot supporting a passive catheter driven by an actuatable drive system or a robot supporting an actuatable catheter having motors and tendons or driven by external forces like a magnetic force); and
(4) a one degree of freedom robot (e.g., robots utilized during a fenestrated endovascular aneurysm repair).

In practice, a medical tool 20 and/or a tool replica 30 may be attachable/detachable from a tool robot 70 (e.g., an endoscope supported by a remote-center-of-motion robot, an ablation catheter disposed within a snake robot, a TEE probe manipulated by a retrofit robotic attachment, a tendon-driven catheter root for vascular navigation or stent deployment) or integrated with a tool robot 70 (e.g., a rigid robot having a distal sawing tool, an ultrasound robot with the transducer integrated into the robot).

Each robotic system 70 may further include a robot controller 72 structurally configured for controlling an actuation of tool robot 71 responsive to pose commands PC instructive of a commanded pose of tool robot 71 within the associated coordinate space as known in the art of the present disclosure.

In practice, a tool robot 70 may incorporate encoder(s) or the like for generating pose data PD informative of a real-time pose of tool robot 71 within an associated coordinate space as known in the art of the present disclosure whereby robot controller 72 is further structurally configured for controlling a generating pose data PD informative of a real-time pose of tool robot 71 within an associated coordinate space as known in the art of the present disclosure. Alternatively or concurrently, component(s) of a spatial tracker 61 as attached to or integrated with a tool robot 70 may provide tracking data TD serving as pose data PD of the tool robot 70.

In practice, when employed, the specific type(s) of robotic system(s) 70 employed by anatomical model medical suite 10 are selected upon the specific type(s) of medical tool(s) 20 and tool replica(s) 30 employed by anatomical model medical suite 10.

Also in practice, a robotic system 70 may be utilized during a pre-operative phase and/or an intra-operative phase of an anatomical model medical procedure as will be further described herein.

Further in practice, alternative to employing a robotic system 70, anatomical model medical suite 10 may be remote communication with a robotic system for receiving pose data PD in real-time as generated by the robotic system 70 and for transmitting pose commands to the robotic system 70 in real-time, and/or the robotic system 70 may employ storage (not shown) (e.g., a database) for an uploading/downloading of pose commands PC previously generated by anatomical model medical suite 10.

Still referring to FIG. 1A, anatomical model medical suite 10 of the present disclosure may further employ a medical workstation 80 for implementing the inventive principles of the present disclosure.

Medical workstation 80 includes or has remote access to a medical procedure controller 90 installed on a computer (not shown). Medical workstation 80 further includes additional components (not shown) customarily associated with a workstation including, but not limited to, a monitor and one or more user input devices (e.g., a keyboards and a mouse).

Medical procedure controller 90 works during a pre-operative phase and/or an intra-operative phase of an anatomical model 40 medical procedure for imaging, diagnosing and/or treating the patient anatomy.

Generally, medical procedure controller 90 controls a position planning and/or a tool guidance of the medical tool 20 relative to the patient anatomy derived from a position planning and/or a tool guidance of the medical tool 20 relative to the anatomical model 40 or a tool replica 30 relative to the anatomical model 40.

By a non-limiting example, the anatomical model medical procedure may pre-operatively involve a manual or robotic tool guidance of the tool replica 30 relative to the anatomical model 40 of the patient anatomy for generating plan data informative of a path planning of the medical tool 20 relative to the patient anatomy, and may intra-operatively involve a manual or robotic tool guidance of the medical tool 20 relative to the patient anatomy in accordance with the plan data.

Additionally, physiologically information may be incorporated into and/or related to the anatomical model 40 to enhance the plan planning and/or tool guidance activities as will be further described herein.

More particularly for a Cox-Maze procedure, pre-operatively, an optical beam of a laser pointer as tracked by the tracking system 60 may be manually guided across an exterior of an anatomical model 40 of a patient heart as a simulation of a catheter ablation of the patient heart whereby the medical procedure controller 90 controls a generation of plan data informative of the simulated catheter ablation of the patient heart. Intra-operatively, the medical procedure controller 90 controls a robotic tool guidance by the robotic system 70 of an ablation catheter across the patient heart in accordance with the plan data to perform the simulated catheter ablation.

Additionally, the anatomical model 40 may be color-coded or texture-coded to identify safe/operable regions and unsafe/inoperable regions of the patient heart for the Cox-Maze procedure whereby the simulated catheter ablation may avoid the unsafe/inoperable regions.

Alternatively, pre-operatively, the optical beam of the laser pointer may be robotically guided by the robotic system 70 across the exterior of the patient heart as the simulation of a catheter ablation of the patient heart whereby the medical procedure controller 90 controls a generation of plan data informative of the simulated catheter ablation of the patient heart.

By further non-limiting example, the anatomical model 40 medical procedure may pre-operatively involve planning information incorporated within the anatomical model 40 of the patient anatomy whereby the planning information is illustrative of a planned path of a medical tool 20 relative to the patient anatomy, and may intra-operatively involve a robotic tool guidance of the medical tool 20 relative to the patient anatomy as a tool replica 30 is manually guided relative to the planned path incorporated within the anatomical model 40 of the patient anatomy.

More particularly for a knee-replacement procedure, pre-operatively, the medical procedure controller 90 controls a position planning of surgical paths across the patient knee within an image of the patient knee as imaged by the imaging system 50 whereby the medical procedure controller 90 generates an anatomical model 40 profile for the manufacturing (e.g., a 3D printing) of an anatomical model 40 of the patient knee incorporating the surgical paths. Intra-operatively, medical procedure controller 90 controls a robotic tool guidance of a robotic saw by a robotic system 70 across the patient knee to form the surgical paths in accordance with a manual tool guidance of a tracked replica saw by the tracking system 60 saw across the surgical paths of the anatomical model 40 of the patient knee or in accordance with a robotic tool guidance by an additional robotic system 70 of the saw across the surgical paths of the anatomical model 40 of the patient knee.

By further non-limiting example, the anatomical model 40 medical procedure may pre-operatively involve a manufacture and/or a coating of an anatomical model 40 of a patient anatomy from material susceptible to a color change in response to an application of a heat or a light to the material, and may intra-operatively involve a robotic tool guidance by a robotic system 70 of a laser pointer relative to the anatomical model 40 of the patient anatomy that mimics a manual tool guidance of a medical tool 20 relative to the patient anatomy whereby heat/light applied by the laser pointer on the anatomical model 40 of the patient anatomy illustrates the manual tool guidance of the medical tool 20 relative to the patient anatomy.

More particularly for a Cox-Maze procedure, pre-operatively, an anatomical model 40 of a patient heart is manufactured or coated from material susceptible to a color change in response to an application of a heat or a light to the material. Intra-operatively, the medical procedure controller 90 controls a robotic tool guidance by a robotic system 70 of a laser pointer relative to the anatomical model 40 of the patient heart that mimics a manual tool guidance of a medical tool 20 relative to the patient heart whereby heat/light applied by the laser pointer on the anatomical model 40 of the patient heart illustrates the manual tool guidance of the ablation catheter across the patient heart.

By further example as exemplary described herein, the anatomical model medical procedure may pre-operatively involve a manual or robotic manipulation of an encoded plane selector with respect to an anatomical model of the patient anatomy whereby medical procedure controller 90 controls a utilization of the plane selector to extract a particular slice from a preoperative 3D image (e.g., ultrasound, MRI, CT, etc.) of the patient anatomy. Alternatively, medical procedure controller 90 may control a utilization of the plane selector position to intra-operatively control a positioning of an imaging device (e.g., control of an angulation of an interventional x-ray c-arm, of a positioning of a robotically controlled TEE probe, or of a focal depth/field-of-view of an ultrasound transducer).

Still referring to FIG. 1A, for purposes of executing the previously described non-limiting exemplary anatomical model medical procedures as well as additional anatomical model medical procedures in accordance with the inventive principles of the present disclosure, medical procedure controller 90 employs an imaging data processing module 91, a tracking data processing module 92 and/or a robot pose data processing module 93.

Imaging data processing module 91 is structurally configured with software/firmware/hardware/circuitry as known in the art of the present disclosure for processing imaging data ID from imaging controller 52 to display relevant 2D/3D images of the subject patient anatomy and associated graphical user interface(s) on the monitor of medical workstation 80. Imaging data processing module 91 may be further structurally configured with software/firmware/hardware/circuitry as known in the art of the present disclosure for facilitating image registration(s) between medical tool(s) 20, tool replica(s) 30, anatomical model(s) 40 and/or tool robot(s) 71 as illustrated in 2D/3D images as needed for the anatomical model medical procedure. Examples of an image registration include, but are not limited to, a manual registration, a land-mark based registration, a feature-based registration and a mechanical registration.

Tracking data processing module 92 is structurally configured with software/firmware/hardware/circuitry as known in the art of the present disclosure for processing tracking data TD to facilitate spatial registration(s) between medical tool(s) 20, tool replica(s) 30, anatomical model(s) 40, medical imager(s) 51 and tool robot(s) 71 as needed for the medical procedure. Examples of a spatial registration include, but are not limited to, a manual registration, a land-mark based registration, a feature-based registration and a mechanical registration.

Robot pose data processing module 93 is structurally configured with software/firmware/hardware/circuitry as known in the art of the present disclosure for processing pose data PD to thereby generate pose commands PC based on a differential between a commanded pose of tool robot 71 within the associated coordinate space and a real-time pose of tool robot 71 within the associated coordinate space as indicated by pose data PD.

For purposes of implementing the inventive principles of the present disclosure, medical procedure controller 90 further employs a model acquisition module 94, a position planning module 95 and/or a tool guidance module 96.

Model acquisition module 94 is structurally configured with software/firmware/hardware/circuitry for facilitating a manufacturing or a selection of an anatomical model 40 of the subject patient anatomy, primarily based on image data ID as processed by imaging controller 52, tracking data TD as processed by tracking controller 62 and/or applicable coordinate system registrations as will be further described herein. Model acquisition module 94 may be further structurally with software/firmware/hardware/circuitry for enhancing an anatomical model 40 as will be further described herein.

Position planning module 95 is structurally configured with software/firmware/hardware/circuitry for facilitating a position planning of a medical tool 20 relative to the subject patient anatomy. The position planning is primarily based on image data ID as processed by imaging controller 52, tracking data TD as processed by tracking controller 62, pose data PD as processed by robot controller 72, and/or applicable coordinate system registrations as will be further described herein.

Tool guidance module 96 is structurally configured with software/firmware/hardware/circuitry for facilitating a tool guidance of a medical tool 20 relative to subject patient anatomy, particularly in accordance with a position planning. The tool guidance is primarily based on tracking data TD as processed by tracking controller 62, pose data PD as processed by robot controller 72, and/or applicable coordinate system registrations as will be further described herein.

Still referring to FIG. 1A, in practice, each modules 91-96 of medical procedure controller 90 may be installed into medical workstation 80 as shown or linked to medical workstation 80. Alternatively, each module of modules 91-96 of medical procedure controller 90 may be partially or fully integrated within one of controller of systems 50, 60 and/or 70, or the modules 91-96 of medical procedure controller 90 may be partially or fully distributed among the controllers of systems 50, 60 and 70.

To facilitate a further understanding of the present disclosure, particularly modules 94-96, the following description of FIG. 1B teaches basic inventive principles of an exemplary anatomical model medical procedure of the present disclosure. From this description, those having ordinary skill in the art will appreciate how to apply the inventive principles of the present disclosure to making and using numerous and varied embodiments of anatomical model medical procedures of the present disclosure.

For clarity purposes in describing the anatomical model medical procedure of FIG. 1, described embodiments for FIGS. 2-8 of a imaging system 50 will be limited to a CT imaging system, of a tracking system 60 will be limited to an electro-magnetic tracking system and of a robotic system 70 will be limited to a snake robotic system. Nonetheless, those having ordinary skill in the art of the present disclosure will appreciate the numerous and varied embodiments of an imaging system, a tracking system and a robotic system applicable to anatomical model medical procedures of the present disclosure.

Referring to FIG. 1B, generally, anatomical model medical procedure 100 of the present disclosure implements an anatomical model acquisition phase 110, a position planning phase 150 and a tool guidance phase 190 for conducting an imaging, a diagnosis and/or a treatment of a subject patient anatomy.

Anatomical model acquisition phase 110 generally provides anatomical model 40 (FIG. 1A) of the subject patient anatomy as will be further exemplary described herein. In practice for anatomical model acquisition phase 110, medical procedure controller 80 may execute any imaging and/or spatial registrations necessary for an acquisition of an anatomical model 40, and/or may implement any type of robotic control necessary for an acquisition of an anatomical model 40.

Position planning phase 150 generally provides a planned position or path of a medical tool 20 (FIG. 1A) relative to the subject patient anatomy that may be derived from a tool guidance of the medical tool 20 or the tool replica 30 (FIG. 1) relative to the anatomical model as will be further exemplary described herein. In practice for position planning phase 150, medical procedure controller 80 may execute any imaging and/or spatial registrations necessary for a position planning of a medical tool 20 relative to the subject patient anatomy, and/or may implement any type of robotic control necessary for a position planning of a medical tool 20 relative to the subject patient anatomy.

Tool guidance phase 190 generally provides a tool guidance of a medical tool 20 (FIG. 1A) relative to the subject patient anatomy that may be derived from a position planning of an anatomical model 40 (FIG. 1) and/or a tool guidance of a tool replica 30 (FIG. 1) relative to the anatomical model 40 as will be further exemplary described herein. In practice for tool guidance phase 190, medical procedure controller 80 may execute any imaging and/or spatial registrations necessary for a tool guidance of a medical tool 20 and/or a tool replica 30, and/or may implement any type of robotic control necessary for a tool guidance of a medical tool 20 and/or a tool replica 30.

In practice, anatomical model medical suite 10 (FIG. 1A) may sequentially and/or concurrently execute phases 120, 130 and 140.

Those having ordinary skill in the art will appreciate that anatomical model medical procedure 100 may execute additional phases not described herein for purposes of directing the description of FIG. 1B exclusively to the inventive principles of the present disclosure.

Still referring to FIG. 1B, anatomical model acquisition phase 110 encompasses an image based model manufacture method 120 for generating a model profile for a manufacturing of an anatomical model from an image of the subject patient anatomy, an image based model selection method 130 for generating a model profile for a selection of an anatomical model from a database of anatomical models, and/or an anatomical model enhancement method 140 applicable to model acquisition methods 120 and 130.

Methods 120 and 130 are patient-specific methods for acquiring an anatomical model. Alternative to methods 120 and 130, an anatomical model may be non-patient-specific, such as, for example, a generic anatomical model manufactured/selected from an anatomical atlas or any type of object physically representative of the patient anatomy. Anatomical model enhancement method 140 is also applicable to non-patient-specific anatomical models.

Still referring to FIG. 1B, image based model manufacture method 120 provides for a 3D printing/rapid prototyping (or other suitable technique) of an anatomical model as known in the art of the present disclosure. In practice, such 3D printing/rapid prototyping of the anatomical model facilitates a manufacture of the anatomical model as a single composite anatomical model or as individual parts that may be post-assembled into the anatomical model.

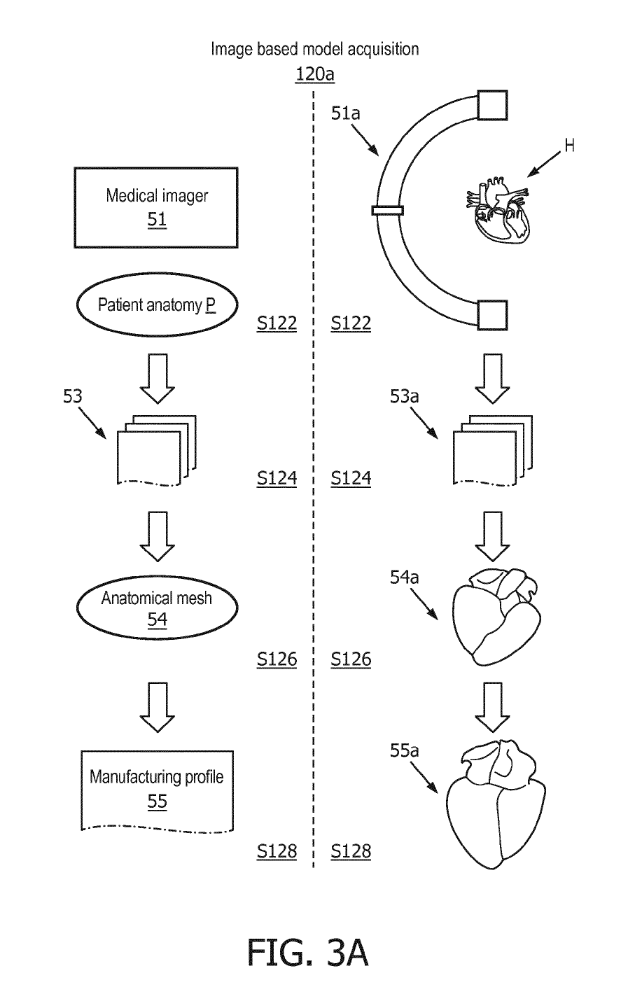

In one embodiment of image based model manufacture method 120, a model acquisition module 94a installed onto or accessible by a medical workstation 90a as shown in FIG. 2A may be operated to execute an image based model manufacture method 120a as shown in FIG. 3A.

Referring to FIG. 3A, image based model manufacture method 120a includes a stage S122 encompassing a pre-operative imaging or an intra-operative imaging by a medical imager 51 (FIG. 1A) of a patient anatomy P to thereby generate image data ID (FIG. 1A) in the form of a 3D image 53 that is processed by imaging controller 52 (FIG. 1A) or model acquisition module 94a (FIG. 2A) during a stage S124 of method 120a. Such processing by imaging controller 53 or model acquisition module 94a involves a segmentation of the 3D image of patient anatomy P and a generation of one or more 3D anatomical meshes 54 of patient anatomy P by model acquisition module 94a during a stage S126 of method 120a whereby an unscaled or a scaled anatomical model 40 (FIG. 1A) of patient anatomy P may be printed using 3D printing/rapid prototyping (or other suitable technique) via a model profile 55 generated by model acquisition module 94a during a stage S128 of method 120a. More particularly, a model profile of the present disclosure delineates dimensional specifications and material composition(s) of an anatomical model derived from the meshes 54 whereby the specifications and material composition(s) are suitable for a printing of the anatomical model.

The result of method 120a is anatomical model 40 being a physical representation of patient anatomy P. In practice, the present disclosure contemplates any level of detail of the physical representation of anatomical model 40a of patient anatomy P that is deemed necessary or minimally required for the performance of anatomical model medical procedure 100 (FIG. 1B).

For example, still referring to FIG. 3A, a C-arm 51a of a CT imaging system may be operated to generate a pre-operative image of a patient heart H to thereby generate image data ID in the form of a 3D image 53a that is processed by model acquisition module 94A (FIG. 2A). Such processing by model acquisition module 94A involves a segmentation of the image and a generation of one or more 3D anatomical meshes 54a of patient heart H whereby an unscaled or a scaled anatomical model 40a of patient heart H may be printed using 3D printing/rapid prototyping (or other suitable technique). The result is anatomical model 40a being a physical representation of patient heart H.

Referring back to FIG. 1B, image based model selection method 130 provides for deriving an anatomical model of the subject patient anatomy by morphing an applicable anatomical model from an anatomical atlas as known in the art of the present disclosure. In practice, such morphing of an applicable anatomical model from an anatomical atlas facilitates a selection of the anatomical model as a single composite anatomical model or as individual parts that may be post-assembled into the anatomical model.

In one embodiment of image based model selection method 130, a model acquisition module 94b installed onto or accessible by workstation 90b as shown in FIG. 2B may be operated to execute image based model selection method 130a as shown in FIG. 3B.

Referring to FIG. 3B, image based model manufacture method 130a includes a stage S132 encompassing a pre-operative imaging or an intra-operative imaging by medical imager 51 (FIG. 1A) of a patient anatomy P to thereby generate image data ID (FIG. 1A) in the form a 3D image series 53 that is processed by imaging controller 52 (FIG. 1A) or model acquisition module 94b (FIG. 2B) during a stage S134 of method 130a. Such processing by imaging controller 52 or model acquisition module 94b involves a measurement of landmarks illustrated within 2D/3D images of subject patient anatomy P to thereby facilitate a generation of a model profile 56 during a stage S136 of method 130a. More particularly, a model profile of the present disclosure delineates dimensional specifications and material composition(s) of an anatomical model suitable for a selection by model acquisition module 94b of a corresponding anatomical model from a database 97 during a stage S138 of flowchart 130 whereby the selected anatomical model may be utilized or morphed into a unscaled or a scaled anatomical model 40 (FIG. 1A) of patient anatomy P.

In practice, database 97 will contain a listing of numerous and various anatomies, particularly from an anatomical atlas, whereby a selected anatomical model may be manufactured or pre-fabricated.

The result of method 130a is anatomical model 40 being a physical representation of patient anatomy P. In practice, the present disclosure contemplates any level of detail of the physical representation of anatomical model 40 of patient anatomy P that is deemed necessary or minimally required for the performance anatomical model medical procedure 100 (FIG. 1B).

For example, still referring to FIG. 3B, a C-arm 51a of a CT imaging system may be operated to generate a pre-operative image or an intra-operative image of a patient heart H to thereby generate image data ID in the form of a 3D image time series 53a that is processed by model acquisition module 94b (FIG. 2B). Such processing by model acquisition module 94b involves a delineation/measurement of landmarks illustrated within 2D/3D images of patient heart H to thereby facilitate a selection of an anatomical model from a model database 97 that is utilized or morphed into a unscaled or a scaled anatomical model 40b of patient heart H. The result is anatomical model 40b being a physical representation of patient heart H.

Referring back to FIG. 1B, anatomical model enhancement method 140 may provide for an incorporation of physiologically-relevant information into the generated/selected anatomical model via methods 120/130 whereby the anatomical model visually highlights such physiologically-relevant information.

More particularly, the term “physiologically-relevant” as described and claims for the present disclosure encompasses any information related to the physiologically of the subject patient anatomy that is relevant to subject anatomical model medical procedure including, but not limited to, organ motion, electrical/vascular pathways, and safe regions for intervention vs. dangerous regions to be avoided.

In practice, the physiologically-relevant information may incorporated into the anatomical model in various ways including, but not limited to:
(1) an illumination by optical projection(s) onto the anatomical model via a laser pointer or laser pointers of different colors, or an optical projector;
(2) a printing and/or a painting of the anatomical model with different textures or colors;
(3) an embedding of LEDs or the like within the anatomical model;
(4) a material composition and/or a coating of the anatomical model that changes color due to heat or light (e.g., thermochromic or photochromic materials); and
(5) the anatomic model may have a flexible material composition whereby active electronic/mechanicals parts may be embedded into, mounted upon or attached to anatomical model to simulate a physiologically motion of the subject patient anatomy (e.g., haptic elements such as vibration motors, haptic screens, piezoelectric elements, etc., for simulating a beating heart motion).

In practice, those having ordinary skill in the art will appreciate manufacturing/coating techniques of the art of the present disclosure applicable to the incorporation of physiologically-relevant information into an anatomical model.

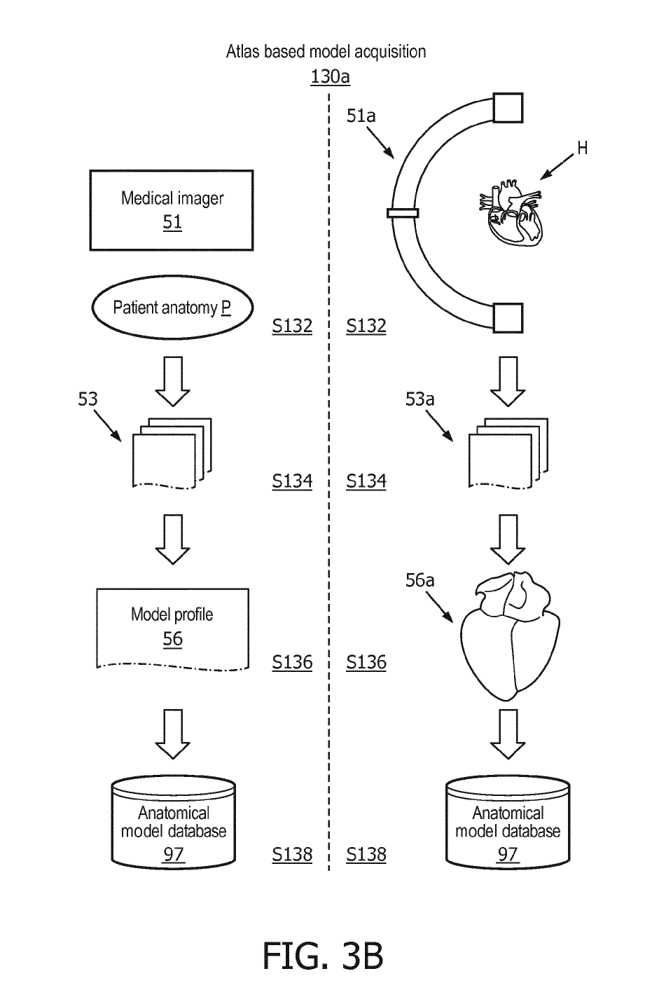

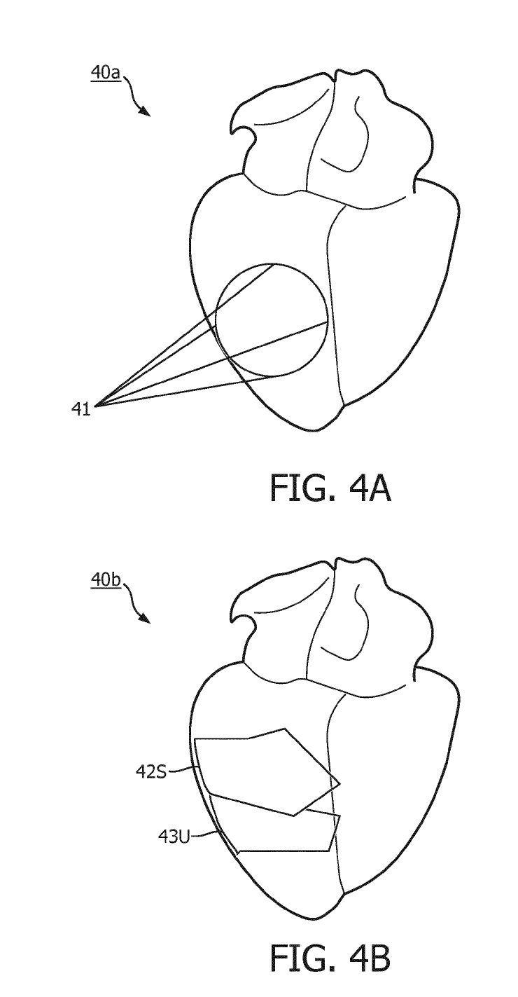

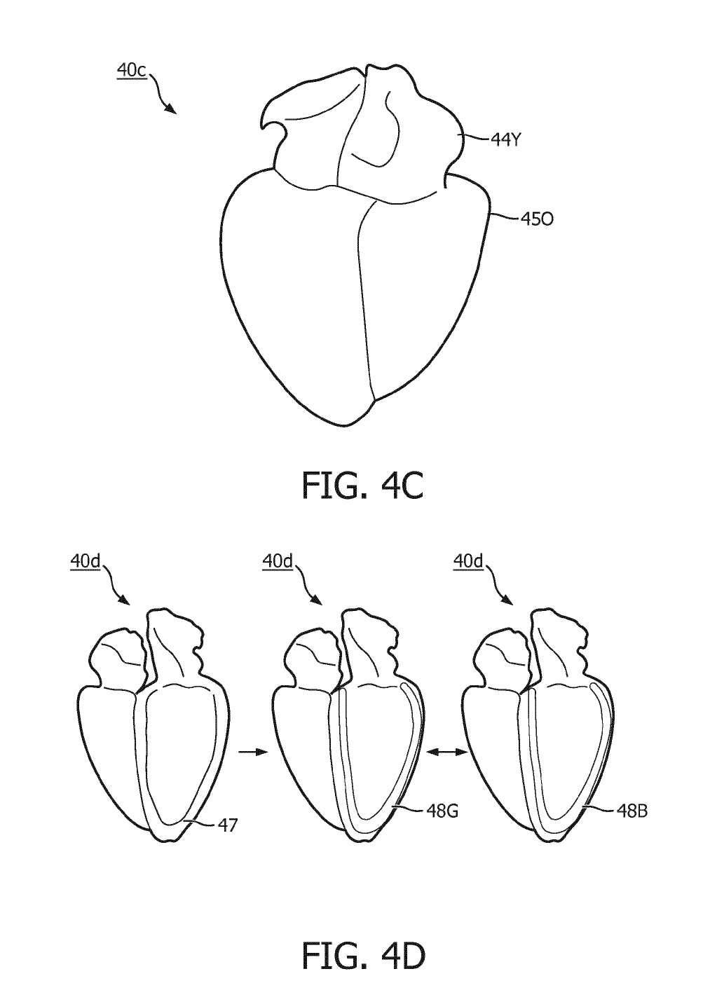

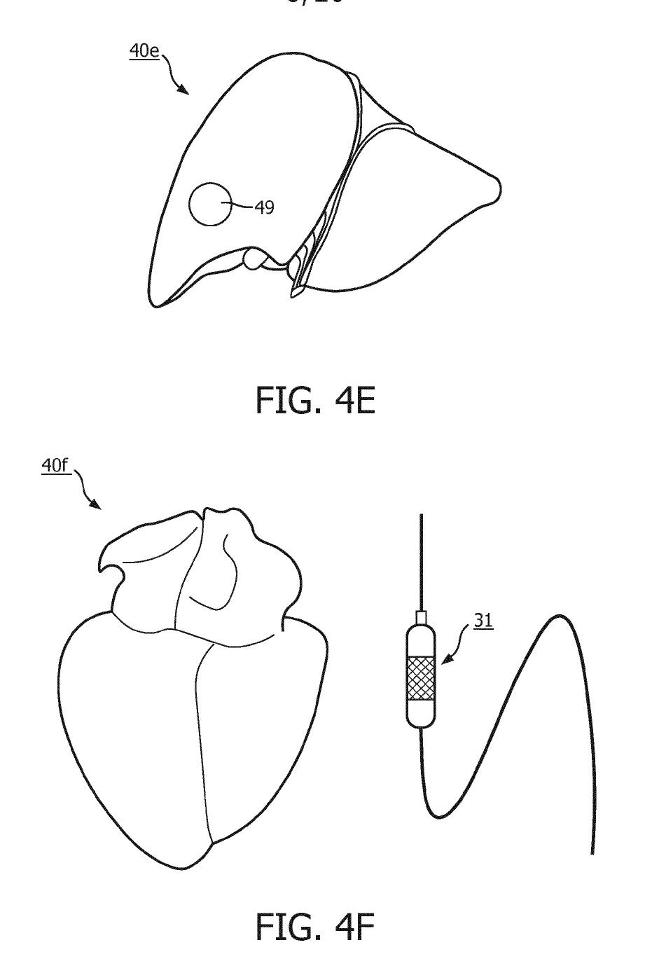

For example, FIG. 4A illustrates an illumination by an optical projection 41 of a laser pointer (not shown) of a safe region of an anatomical model 40a relevant to the medical procedure.

By further example, FIG. 4B illustrates a printing of an anatomical model 40b with different textures or colors for a safe region 42S and an unsafe region 43U relevant to the medical procedure.

By further example, FIG. 4C illustrates a printing of an anatomical model 40c with different colors such as for example, a yellow color for an aorta 44Y, an opaque color for ventricles 45O and a red color for arties/veins (not shown) traversing the ventricles 45O.

By further example, FIG. 4D illustrates a printing of a groove 47 within an anatomical model 40d whereby a LED 47 is inserted within groove 47 and LED 48 is switched between a green color 48G and a blue color 48B to highlight a dynamic physiology on anatomical model 40d.

For the four (4) examples of FIGS. 4A-4D, a haptic element (not shown) may be embedded/mounted/attached with the anatomical model to simulate a beating of the patient heart.

Referring back to FIG. 1B, anatomical model enhancement method 140 may provide for an incorporation of procedural-relevant information into the generated/selected anatomical model via methods 120/130 whereby the anatomical model visually highlights such procedural-relevant information. More particularly, the term “procedural-relevant” as described and claims for the present disclosure encompasses any information related to position planning and tool guidance of a medical tool and/tool replica relative to the anatomical model and/or the subject patient anatomy including, but not limited to, locations and orientations of implantable devices within the anatomical model, reference position(s) of the medical tool(s) 20 relative to the anatomical model and path planned location(s) of the medical tool(s) 20 relative to the anatomical model.

In practice, the procedural-relevant information may be incorporated into the anatomical model in various ways including, but not limited to, printing or integration of one or more physical features (e.g., a hook, a hole a clip, etc.) into the anatomical model.

In one embodiment, the procedural-relevant information may be incorporated as a target position onto or into the anatomical model. For example, FIG. 4E illustrates an incorporation of a target including an entry point into a patient liver identified as a target hole 49 into an anatomical model 40e of a patient liver.

Referring back to FIG. 1B, anatomical model enhancement method 140 may provide for a manufacture of tool replica 30 as a model of a medical tool. Examples of such manufactured tool replicas include, but are not limited to, the medical tool itself, stents, guidewires, and implantable devices (e.g., valves, clips, screws, rods, etc.), a pointer, a finger, a laser pointer, a model of a c-arm or a TEE probe.

In practice, a tool replica 30 may be a model of the medical tool in a undeployed, semi-deployed or fully deployed state and in various positions.

For example, FIG. 4F illustrates a model 31 of an undeployed aortic valve deployment system that was manufactured with or selected to be utilized with a hollow anatomical model 40f of a patient heart. In practice, those having ordinary skill in the art will appreciate techniques of the art of the present disclosure applicable for manufacturing a replica of a medical tool.

Referring back to FIG. 1B, any enhancements to an anatomical model as previously described herein may be incorporated with a model profile as applicable to a manufacturing of the anatomical model or within a model profile as applicable to a selection of an anatomical model.

Furthermore, in practice, anatomical model medical procedure 100 may be executed over numerous sequential segments of time whereby the physical state of the patient anatomy may change from time segment to time segment (e.g., a Cox-Maze procedure). In practice, methods 120 and/or 130 as optionally enhanced by method 140 may therefore be executed for each segment of time to thereby generate/select multiple versions of the anatomical model with each anatomical model physically representing the patient anatomy during a corresponding segment of time.

Still referring to FIG. 1B, position planning phase 150 encompasses a non-model based position planning method 160 for a position planning of a medical tool 20 (FIG. 1A) relative to the subject patient anatomy from an imaging of the subject patient anatomy. Position planning phase 150 further encompasses a pre-operative and intra-operative model base position planning methods 170 and 180 for position planning of a medical tool 20 relative to the subject patient anatomy from a tool guidance of the medical tool 20 relative to an anatomical model 40 (FIG. 1A) or a tool replica 30 (FIG. 1A) relative to an anatomical model 40 (FIG. 1A).

Generally, the position planning involves a plan of a “procedural positioning” broadly encompassing any translational motion, any rotational motion and any pivotal motion of a medical tool 20 or a tool replica 30 within a geometric space leading to a location on the subject patient anatomy and/or any translational motion, any rotational motion and any pivotal motion of a medical tool 20 or a tool replica 30 spatially or contiguously traversing an exterior and/or an interior of the subject patient anatomy for purposes of diagnosing and/or treating the subject patient anatomy

In practice, the plan may be expressed as a spatial representation including, but not limited to:
(1) a plane (e.g., cutting planes for orthopedic procedures, such as knee or hip replacement surgery);
(2) a path or a set of paths (e.g., for Cox-Maze procedure on the heart or EP);
(3) an area (e.g., landing zone in a vessel for stent of graft deployment or a tumor area);
(4) safety zones (e.g., vasculature, sensitive structures in the brain, etc.);
(5) dots (e.g., insertion points for needle biopsy or needle ablation); and
(6) rings for delineating target branches of vessels or airways.

Still referring to FIG. 1B, position planning phase method 160 provides for position planning based on an imaging of the subject patient anatomy as known in the art of the present disclosure.

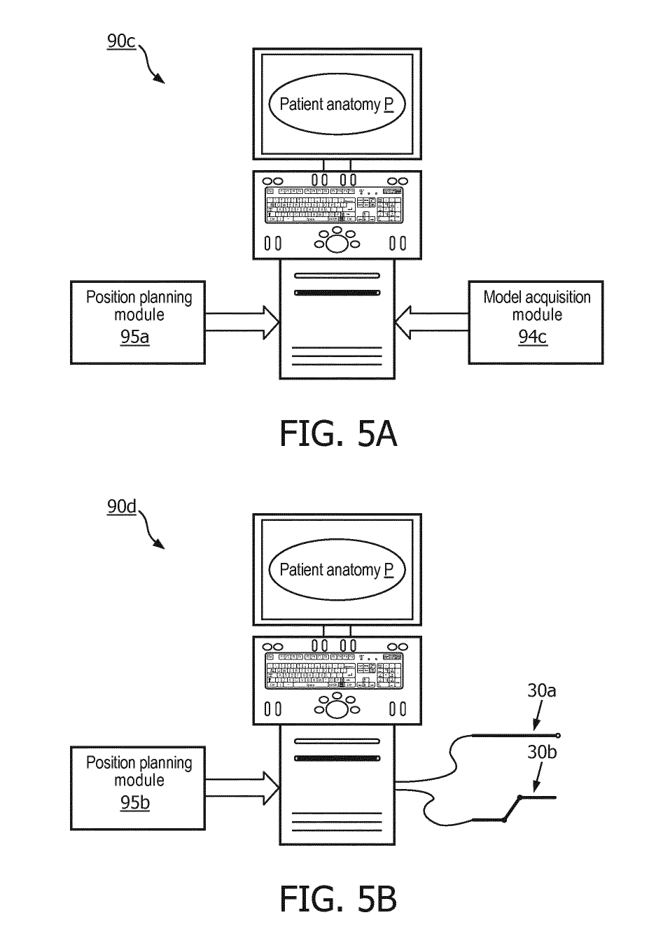

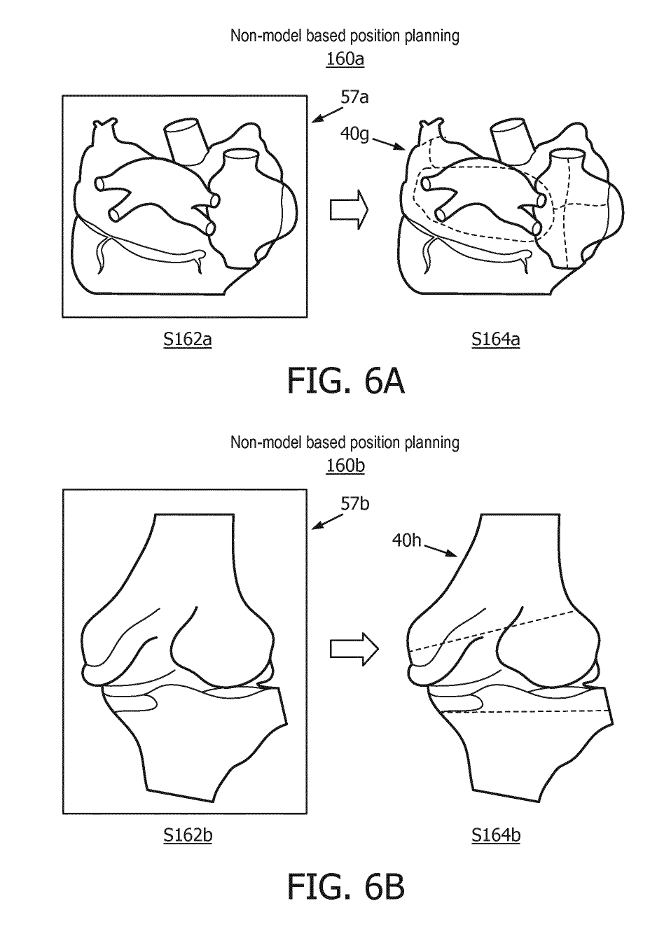

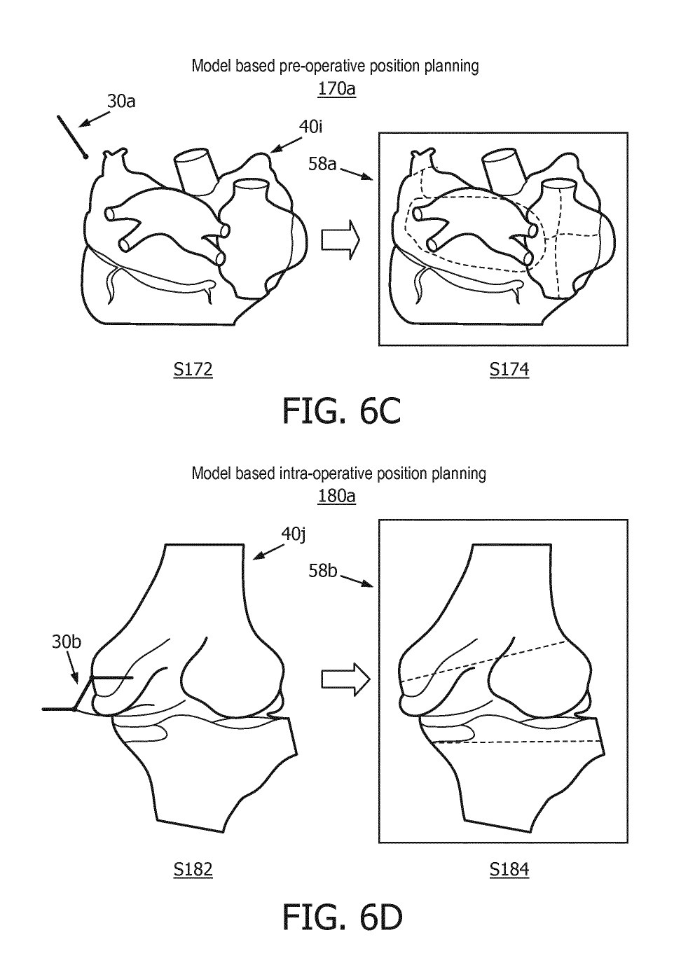

In one embodiment of position planning phase method 160, a position planning module 95a installed onto or accessible by a medical workstation 90c as shown in FIG. 5A may be operated to execute a non-model based position planning methods 160a and 160b as respectively shown in FIGS. 6A and 6B.

Referring to FIG. 6A, a stage S162a of method 160a encompasses a display of an imaging 57a of a patient heart whereby position planning techniques as known in the art of the present disclosure are implemented via graphical user interfaces to render a planned path of a medical tool 20 relative to the patient heart. A stage S164a of method 160a encompasses a storage of the planned path for execution of tool guidance phase 190, or alternatively encompasses an incorporation of the planned path within a model profile of method 120 or within a model profile of method 130 whereby an anatomical model 40 may be acquired with planned path features incorporated within the anatomical model 40.

More particularly, FIG. 6A illustrates anatomical model 40g having planned path features for a Cox-Max procedure embedded within the wall of a patient heart as symbolized by the dashed lines. As previously described herein, the embedding of the planned path may involve a color-coded or texture-coded scheme for differentiating the planned path from the rest of the anatomical model, or may involve a 3D printing of the anatomical wall with grooves representative of the planned path within the wall of the patient heart whereby LEDs may or may not be embedded in the grooves.

Similarly, referring to FIG. 6B, a stage S162b of method 160b encompasses a display of an imaging 57b of a patient knee whereby position planning techniques as known in the art of the present disclosure are implemented via graphical user interfaces to render a planned path of a medical tool 20 relative to the patient knee. A stage S164b of method 160b encompasses a storage of the planned path for execution of tool guidance phase 190, or alternatively encompasses an incorporation of the planned path within a model profile of method 120 or within a model profile of method 130 whereby an anatomical model 40 may be acquired with planned path features embedded within, mounted onto or attached to the anatomical model 40.

More particularly, FIG. 6B illustrates a display of an imaging 57b of a patient knee whereby anatomical model 40h having planned path features for a knee replacement surgery are embedded within the bone of the patient knee as symbolized by the dashed lines. As previously described herein, the embedding of the planned path may involve a color-coded or texture-coded scheme for differentiating the planned path from the rest of the anatomical model, or may involve a 3D printing of the anatomical wall with grooves representative of the planned path within the wall of the patient heart whereby LEDs may or may not be embedded in the grooves.

Referring back to FIG. 1B, model based position planning methods 170 and 180 provide for position planning based an anatomical model in accordance with the inventive principles of the present disclosure.

In one embodiment of model based position planning methods 170 and 180, a position planning module 95b as installed onto or accessible by a medical workstation 90d as shown in FIG. 5B may be operated to execute a pre-operative based position planning method 170a as shown in FIG. 6C and/or an intra-operative based position planning method 180a as shown in FIG. 6D. For methods 160a and 170a, medical workstation 90d provides a tool replica 30 in the form of a laser pointer 30a including tracking sensor(s) (e.g., a FORS sensor), and an additional tool replica 30 in the form of an encoded robot arm 30b. Laser pointer 30a and encoded robot arm 30b facilitates a simulated procedural positioning of a medical device 20 on the subject patient anatomy.

Referring to FIG. 6C, prior to execution of pre-operative position planning method 170a, a tracking registration of an anatomical model 40i of a patient heart to a pre-operative image 58a of the patient heart is performed via any suitable registration technique as known in the art of the present disclosure, such as, for example, by use of a laser pointer 30a identifying points on anatomical model 40i that are illustrated in the pre-operative image 58a of the subject patient anatomy. Upon completion of the tracking registration, a stage S172 of method 170a encompasses a procedural positioning of laser pointer 30a on the anatomical model to mark a simulated location or a traversal of medical tool 20 on the subject patient anatomy during tool guidance phase 190.

A stage S174 of method 170a encompasses an overlay of the planned path on the pre-operative image 58a of the patient heart as symbolized by the dashed lines for a Cox-Max procedure.

Referring to FIG. 6D, prior to an execution of an intra-operative position planning method 180a, a registration of encoded robot arm 30b to an intra-operative image 58b of an anatomical model 40j of a patient knee via tracking system 50 is performed via any suitable registration techniques as known in the art of the present disclosure, such as, for example, a registration involving a placement of tracking sensors onto robot arm 30b. Upon completion of the tracking registration, a stage S182 of method 180a encompasses a procedural positioning of rigid robot arm 30b on the anatomical model to mark a simulated location or a traversal of rigid robot arm 30b on the subject patient anatomy during tool guidance phase 190.

A stage S184 of method 180a encompasses an overlay of the planned path on the intra-operative image 58b of the patient knee as symbolized by the dashed lines for knee replacement surgery.

Referring back to FIG. 1B, tool guidance phase 190 encompasses a non-model based tool guidance method 200 for executing a pre-operative planned path of a medical tool 20 (FIG. 1A) relative to the subject patient anatomy, and model based pre-operative and intra-operative tool guidance method 210 and 220 for guiding a medical tool 20 relative to the subject patient anatomy.

Generally, the tool guidance involves tool guidance module 96 (FIG. 1A) generating pose commands PC instructive of a commanded pose of tool robot 71 (FIG. 1A) within an associated coordinate space in accordance with pre-operative planned path, a procedural positioning of a medical tool 20 relative to the subject patient anatomy or a procedural positioning of a tool replica 30 (FIG. 1A) relative to an anatomical model 40 (FIG. 1A) of the subject patient anatomy. The tool guidance may further involve tool guidance module 96 generating pose commands PC instructive of a commanded pose of a medical imager 51 (FIG. 1) within an associated coordinate space in accordance with a procedural positioning of a tool replica 30 relative to an anatomical model 40 of the subject patient anatomy.

Still referring to FIG. 1B, non-model based tool guidance method 200 provides for a tool guidance of a medical tool 20 in accordance with a pre-operative planned path, particularly a pre-operative planned path generated by method 160, 170 and 180.

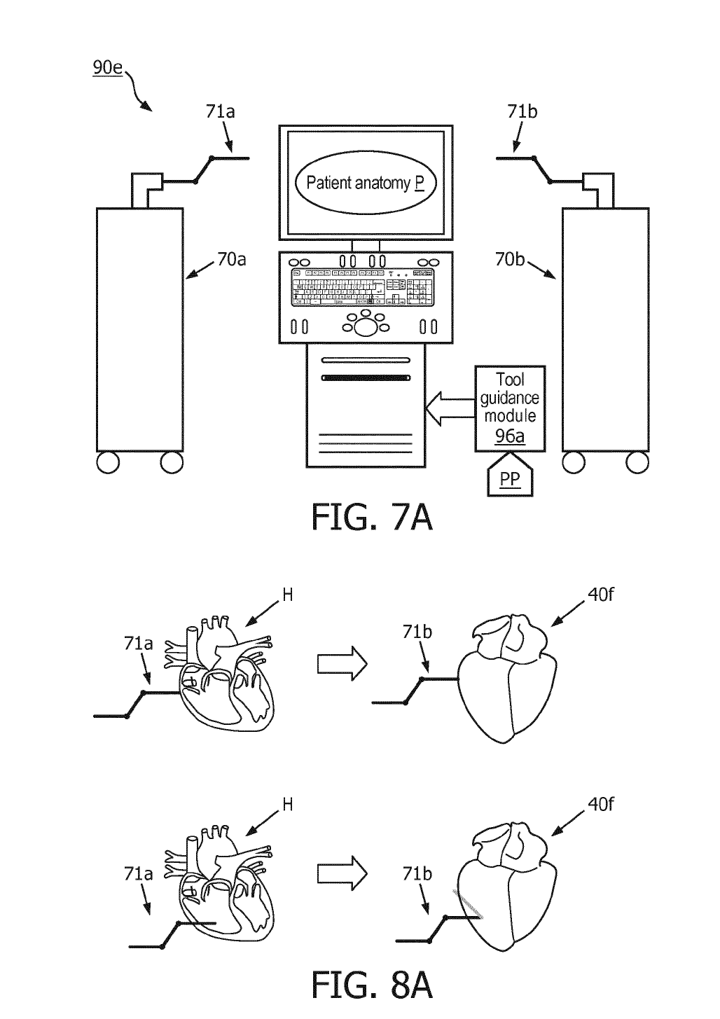

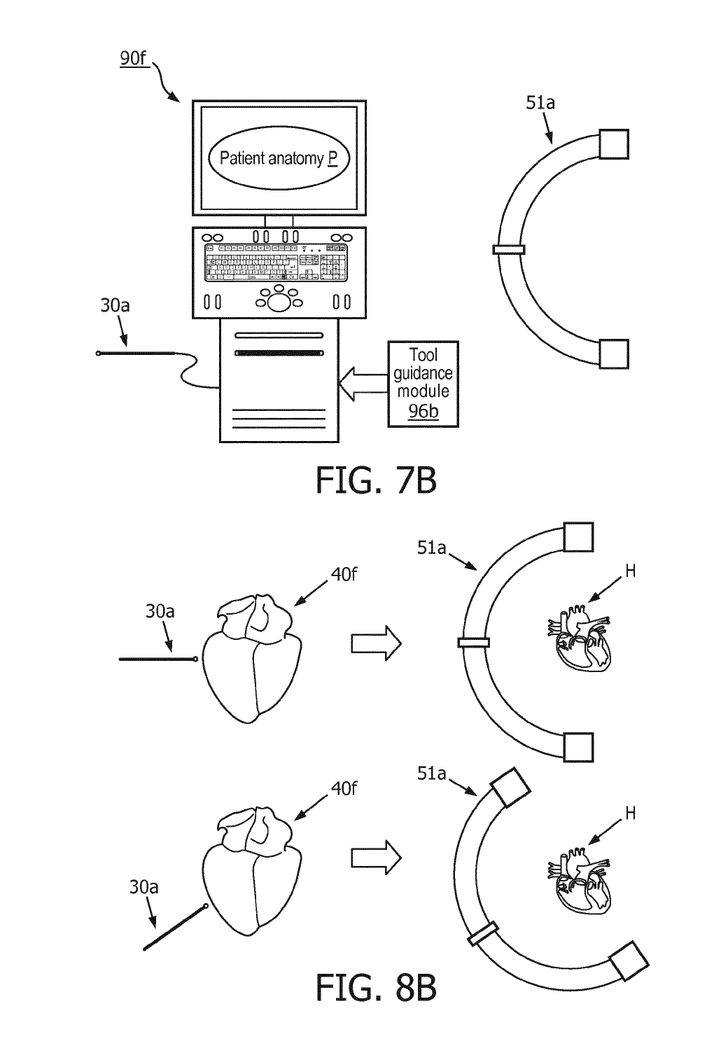

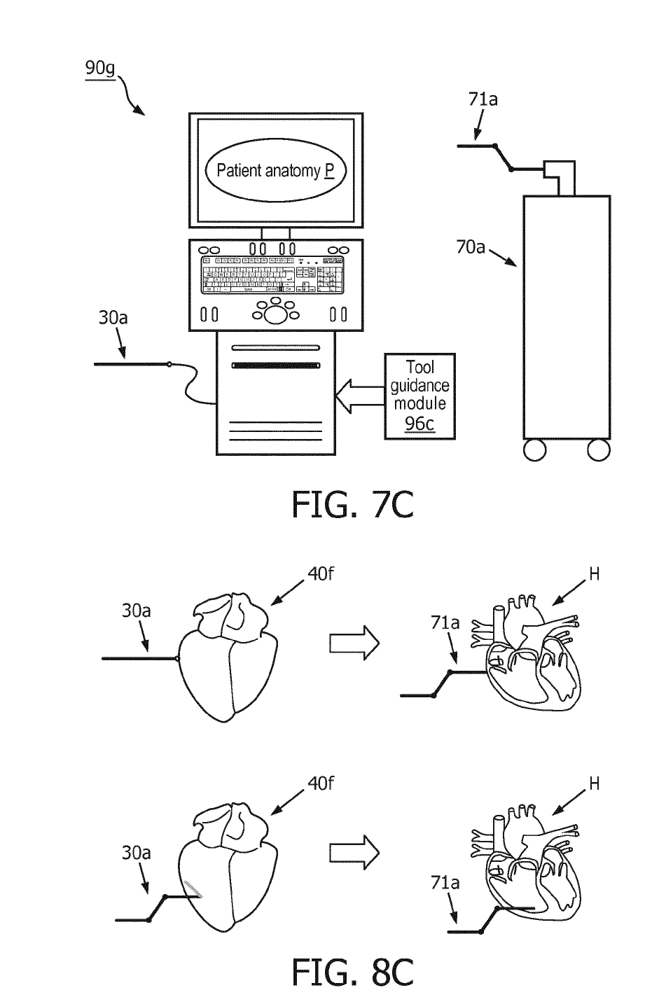

In one embodiment of non-model based tool guidance method 200, a tool guidance module 96a installed onto or accessible by a medical workstation 90e as shown in FIG. 7A may be operated to execute a non-model based tool guidance method 200.

Prior to an execution of this embodiment of method 200, a robotic system 70b is registered to a tracking system 60 (FIG. 1A) if utilized in the position planning phase 150. Such registration is perform in accordance with known registration techniques of the art of the present disclosure, such as, for example, a registration involving of a placement of tracking sensors on a robotic arm of robotic system 70b. Otherwise, robotic system 70b will include encoded pre-operative path planned data if robotic system 70b or equivalent robotic system was utilized in the position planning phase 150.

Method 200 initially encompasses robot tool 71a supporting a medical tool 20 (not show) (e.g., an ablation catheter) being inserted into the patient or positioned in proximity of that patient anatomy in dependence upon a starting point of the pre-operative planned path.

Method 200 thereafter encompasses either tool guidance module 96a transforming the tracked pre-operative planned path into the robotic coordinate system and communicating pose commands PC to robotic system 70b whereby robot tool 71a follows the pre-operative path as illustrated in a virtual overlay of robot tool 71a on a pre-operative image of the subject patient anatomy, or robotic system 70b processing the encoded pre-operative path planned data whereby robot tool 71a follows the pre-operative path as illustrated in a virtual overlay of robot tool 71a on a pre-operative image of the subject patient anatomy.

Method 200 may further incorporate a robotic system 70b for positioning a robot tool 71b relative to an anatomical model 40f to thereby provide additional feedback of the procedural positioning of robot tool 71a relative to the subject patient anatomy.

For example, a robotic system 70b has a robot tool 71b supporting a tool replica 30 (FIG. 1A) (e.g., a laser pointer) whereby the robotic system 70b is registered to robotic system 70a. Robot tool 71b is positioned at a starting point of the pre-operative planned path relative to the anatomical model whereby robotic system 70a provides pose data PD of robot tool 71a to tool guidance module 96a, which transforms the pose data PD into pose commands for robotic system 70b to procedurally position the tool replica 30 supported by robot tool 71b relative to anatomical model 40f.

As a result, as shown in FIG. 8A, the tool replica 30 as supported by robot tool 71b provides feedback of the processing positioning of robot tool 71a relative to the subject patient anatomy.

Referring back to FIG. 1B, model based pre-operative tool guidance method 210 provides for a tool guidance of a medical imager 51 (FIG. 1A) serving as a medical tool for diagnosing the subject patient anatomy. For example, the medical imager 51 maybe any medical imager including, but not limited to, an CT c-arm, a robotically controlled Ultrasound transducer (e.g., a TEE probe) or a robotically controlled endoscope. A properly registered tool replica 30 (FIG. 1A) may then be utilized to transform any positioning of the tool replica 30 relative to anatomical model into a procedural positions of the medical imager 51 relative to the subject patient anatomy.

For example, in one embodiment of method 210, a tool guidance module 96b installed onto or accessible by a medical workstation 90f as shown in FIG. 7B may be operated to execute a method 210. In this embodiment, a tracked pointer 30a is used to identify a location or plane of interest relative to an anatomical model 40f as shown in FIG. 8B and then an X-ray arm 51a is oriented to provide the desired positional view of a patient heart H.

Alternatively, a model of CT c-arm 51a may be a tracked pointer whereby a user manipulates the model of CT c-arm 51a into an intended position and orientation relative to anatomical model and then CT c-arm 51a takes on a corresponding position and orientation relative to the subject patient anatomy.

By further example, the medial imager 51 may be a robotically-controlled TEE probe whereby a tracked pointer serving as a tool replica of a head of the TEE probe may be positioned relative to an anatomical model of the subject patient anatomy and the robotically-controlled TEE probe will move to a corresponding position relative to the anatomical model. Alternatively, the tracked pointer may be orthogonally moved relative to the anatomical model of the subject patient anatomy to represent a plane selector that is used to pick a 2D cross-section of the 3D ultrasound volume.

In another embodiment of the method 210, medical imager 51 is controlled manually by the user. For example, angles of CT c-arm 51a are controlled by a user via imaging controller 52 (FIG. 1A) based on knowledge of values of those angles. In this embodiment, tool guidance module 96b communicates via a display the positioning of the medical imager 51 relative to the subject patient anatomy as derived from the positioning of the registered tool replica 30 relative to the anatomical mode. For example, tool guidance module 96b informs the user via the display of the desired position and orientation of CT c-arm 51a relative to patient heart H as derived by the positioning to the tracked pointer 30a relative to anatomical model 40f as shown in FIG. 8B, and the user may operate imaging controller 52 to maneuver CT c-arm 51a to the desired position and orientation relative to patient heart H.

Referring back to FIG. 1B, model based intra-operative tool guidance method 220 provides for a tool guidance of medical tool for diagnosing and/or treating the subject patient anatomy.

In one embodiment of method 220, a tool guidance module 96c installed onto or accessible by a medical workstation 90g as shown in FIG. 7C may be operated to execute method 220. A pre-requisite to the execution of method 220 is:
(1) a registration of an anatomical model 30 (FIG. 1A) to pre-operative images of the subject patient anatomy as known in the art of the present disclosure;
(2) registration of a tracking system 60 (FIG. 1A) to the subject patient anatomy within the operating room as known in the art of the present disclosure, and
(3) a utilization of the tracking system 60 to register the subject patient anatomy to the pre-operative images of the subject patient anatomy as known in the art of the present disclosure, which implicitly registers the anatomical model to the patient anatomy.

Alternatively, an intra-operative imaging system may be utilized for registering the subject patient anatomy to the pre-operative images of the subject patient anatomy, or for generating an intra-operative image illustrative of both the anatomical model and the subject patient anatomy.

Subsequent to the registrations, method 220 encompasses a medical tool as supported by robot tool 71a to be inserted into the patient or positioned in proximity of the medical site. Thereafter, a laser pointer 30a is positioned on the anatomical model to mark a path or a location where robot tool 71a should be positioned relative to a patient heart H. Tool guidance module 96c transforms the desired path or location to the coordinate frame of robotic system 70b and controls a communication of pose commands PC to robot controller 72, which converts the pose commands into actuation signals for robot tool 71b whereby robot tool 71b follows the path relative to patient heart H as defined by the path of laser point 30a relative to anatomical model 40f as shown in FIG. 8C. Imaging system 30 displays the position of the medical tool in a virtual representation of the patient anatomy.

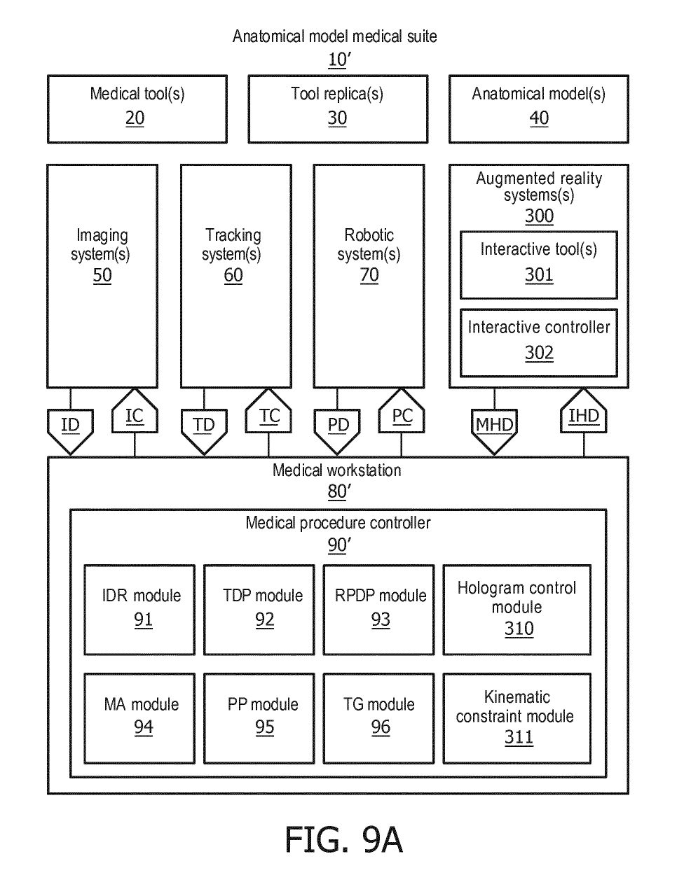

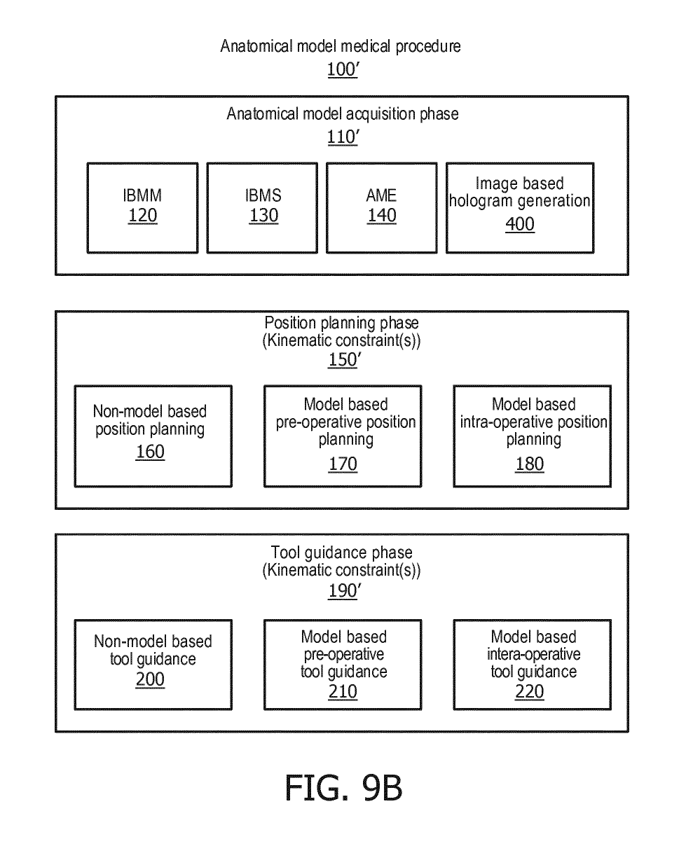

To facilitate a further understanding of the present disclosure, the following description of FIG. 9A teaches basic inventive principles of an exemplary anatomical model medical suite of the present disclosure incorporating the anatomical model(s) 40 (FIG. 1A) and/or the tool replica(s) 30 (FIG. 1A) as holograms. From this description, those having ordinary skill in the art will appreciate how to further apply the inventive principles of the present disclosure to making and using numerous and varied embodiments of anatomical model medical suites of the present disclosure incorporating the anatomical model(s) and/or the tool replica(s) as holograms.

Referring to FIG. 9A, an anatomical model medical suite 10′ of the present disclosure employs one or more medical tools 20, one or more optional tool replicas 30, one or more optional imaging system(s) 50, one or more optional tracking system(s) 60 and one or more optional robotic system(s) 70 as previously described herein for the anatomical model medical suite 10 shown in FIG. 1A.

Still referring to FIG. 9A, anatomical model medical suite 10′ further employs one or more augmented reality system(s) 300 for generating one or more holographic anatomical models 40 and/or one or more optional holographic tool replicas 30 as known in the art of the present disclosure.

In practice, an augmented reality system 300 may be a standard component of anatomical model medical suite 10′ employable for one or more anatomical model medical procedures performed by anatomical model medical suite 10′, or selectively acquired for a specific anatomical model medical procedure to be performed via anatomical model medical suite 10′.

Further in practice, an augmented reality system 300 includes one or more interactive tools 301 for facilitating a user interaction with a 2D or 3D hologram of an anatomical model 40 and/or a tool replica 30 as known in the art of the present disclosure. Examples of an interactive tool 301 include, but are not limited to:
1. a pointer (encoded, tracked, robotic);
2 a finger/hand/gesture tracking device (a camera-based gesture recognition);
3. a plane selector (encoded, tracked, robotic);
4. a voice recognition device;
5. a user's position tracking device (e.g., physical position in the room, gaze, head position);
6. a robot;
7. a 3D printed anatomical model of the same hologram;
8. a replica of an imaging system 50; and
9. a marker-based tracking object.

Each augmented reality system 300 furthers includes an interactive controller 302 structurally configured for controlling the user interaction with a 2D hologram or a 3D hologram of an anatomical model 40 and/or a tool replica 30 as known in the art of the present disclosure. More particularly, interactive controller 302 controls a holographic display of the anatomical model 40 and/or tool replica 30 as indicated by imaged hologram data IHD from a hologram control module 310 of a medical procedure controller 90′ as will be further exemplary explained herein. From the holographic display, interactive controller 302 communicates manipulated hologram data MHD to hologram control module 310 to thereby inform hologram control module 310 of any path planning and/or tool guidance aspects of the user interaction with the 2D hologram or the 3D hologram of an anatomical model 40 and/or a tool replica 30.

In practice, the specific type(s) of augmented reality system(s) 300 employed by anatomical model medical suite 10′ are selected based upon the specific type(s) of anatomical model medical procedure(s) to be performed via anatomical model medical suite 10′.

Also in practice, augmented reality system 300 may be utilized during a pre-operative phase and/or an intra-operative phase of anatomical model medical procedure as will be further described herein.

Further in practice, alternative to employing augmented reality system 300, anatomical model medical suite 10′ may be in remote communication with augmented reality system 300 for receiving manipulated hologram data MHD in real-time as generated by the augmented reality system 300 and/or may employ storage (not shown) (e.g., a database) for an uploading/downloading of manipulated hologram data MHD previously generated by the augmented reality system 300.

Still referring to FIG. 9A, anatomical model medical suite 10′ of the present disclosure may further employ a medical workstation 80′ for implementing the inventive principles of the present disclosure.

Medical workstation 80′ includes or has remote access to a medical procedure controller 90′ installed on a computer (not shown). Medical workstation 80′ further includes additional components (not shown) customarily associated with a workstation including, but not limited to, a monitor and one or more user input devices (e.g., a keyboards and a mouse).

Medical procedure controller 90′ works during a pre-operative phase and/or an intra-operative phase of an anatomical model 40 medical procedure for imaging, diagnosing and/or treating the patient anatomy.

Generally, as with medical procedure controller 90 (FIG. 1A), medical procedure controller 90′ controls a position planning and/or a tool guidance of the medical tool 20 relative to the patient anatomy derived from a position planning and/or a tool guidance of the medical tool 20 relative to the anatomical model 40 or a tool replica 30 relative to the anatomical model 40. Similar to medical procedure controller 90, medical procedure controller 90′ employs modules 91-96 (FIG. 1A). In addition medical procedure controller 90′ employs a hologram control module 310 and an optional kinematic constraint module 311.

Hologram control module 310 is structurally configured with software/firmware/hardware/circuitry for generating imaged hologram data IHD from imaging data ID information of an illustration of the anatomy of interest of the patient, pre-operatively o intra-operatively. In practice, hologram control module 310 executes a segmentation technique as known in the art of the present disclosure to thereby segment the patient anatomy of interest from imaging data ID and communicate the segmented patient anatomy as imaged hologram data IHD.

Hologram control module 310 is structurally configured with software/firmware/hardware/circuitry for processing manipulated hologram data MHD to reflect any user interaction with holographic anatomical model(s) 40 and/or tool replica(s) 30 to thereby communicate such processed of manipulated hologram data MHD to imaging system(s) 50 for display purposes, for planning module 95 for planning purposes and/or to tool guidance model 96 for guidance purposes. Examples of such processing include, but are not limited to:
1. an updating of the holographic anatomical model(s) 40 and/or the holographic tool replica(s) 30 to reflect any user edits of the holographic anatomical model(s) 40 and/or the holographic tool replica(s) 30 (e.g., a cropping of a holographic anatomical model);
2. an updating of a display of imaging data ID a on a monitor separate from imaging system(s) 50 to reflect any user edits of the holographic anatomical model(s) 40 and/or the holographic tool replica(s) 30 (e.g., a cropping of an ultrasound image, rotation of a pre-operative CT image); and
3. control imaging parameters of imaging system(s) 50 (e.g., a position of a C-arm gantry, a focus of an ultrasound transducer, etc.).

Alternatively in practice, interactive controller 302 may employ hologram control module 310, or hologram control module 310 may be distributed between medical procedure controller 90′ and interactive controller 302.

Kinematic constraint module 311 is structurally configured with software/firmware/hardware/circuitry for indicating motion constraints of an imaging system 50, a tool replica 30 of an imaging system 50 and/or an interactive tool 301 of augmented reality system 300 (i.e., kinematic devices) as understood by those having ordinary skill in the art of the present disclosure. Examples of such motion constraints include, but are not limited to:
1. interference anatomy of a patient surrounding the anatomy of interest as ascertained in practice;
2. interference with objects and/or medical staff surrounding the patient and/or the imaging system/replica as ascertained in practice;
3. an unattainable position of the kinematic device as ascertained in practice;
4. a known sub-optimal view of the patient anatomy and/or medical tool 20 as ascertained in practice;
5. an avoidance of certain positions limit radiation exposes to the patient and/or the medical staff as ascertained in practice;
6. therapeutic dose information as ascertained in practice; and
7. operation specified constraints.

In practice, kinematic constraint module 311 is in communication with an imaging system 50, a tool replica 30 of an imaging system 50 and/or an interactive tool 301 of augmented reality system 300 to ascertain a position thereof and executes any feedback technique known in the art of the present disclosure including, but not limited to:
1. providing haptic feedback (e.g., a vibration) whenever a position of a kinematic device violates a constraint or approaches an unfavorable/unattainable position with respect to the anatomical model or the patient anatomy;
2. controlling an increasing mechanical resistance of the kinematic device as the kinematic device approaches an unfavorable or an unattainable position with respect to the anatomical model or the patient anatomy;
3. controlling an activation/a deactivation of a visual indicator (e.g., LED); to indicate favorable/attainable positions of the kinematic device with respect to the anatomical model or the patient anatomy (e.g., LED is green) or unfavorable/unattainable positions of the kinematic device with respect to the anatomical model or the patient anatomy (e.g., LED is red);
4. controlling a display of an anatomical model and kinematic device whereby visual feedback provides an indication of favorable/attainable positions of the kinematic device with respect to the anatomical model or the patient anatomy (e.g., the kinematic device is green) or unfavorable/unattainable positions of the kinematic device with respect to the anatomical model or the patient anatomy (e.g., the kinematic device is gray/red and/or flashes); and
5. controlling auditory feedback, particularly via an augmented reality headset.

Concurrent or alternative to kinematic constraint module 311, an imaging system 50, a tool replica 30 of an imaging system 50 and/or an interactive tool 301 of augmented reality system 300 (i.e., kinematic device) may be manufactured/retrofitted to provide mechanical feedback as known in the art of the present disclosure including, but not limited to an incorporation of physical stops and/or mechanical resistance that prevents/impedes the kinematic device from moving to an unfavorable position.

To facilitate a further understanding of the present disclosure, particularly modules 310 and 311 (FIG. 9A), the following description of FIG. 9B teaches basic inventive principles of an exemplary anatomical model medical procedure of the present disclosure incorporating the anatomical model(s) 40 (FIG. 9A) and/or the tool replica(s) 30 (FIG. 1A) as holograms. From this description, those having ordinary skill in the art will appreciate how to apply the inventive principles of the present disclosure to making and using numerous and varied embodiments of anatomical model medical procedures of the present disclosure incorporating anatomical model(s) and/or tool replica(s) as holograms.

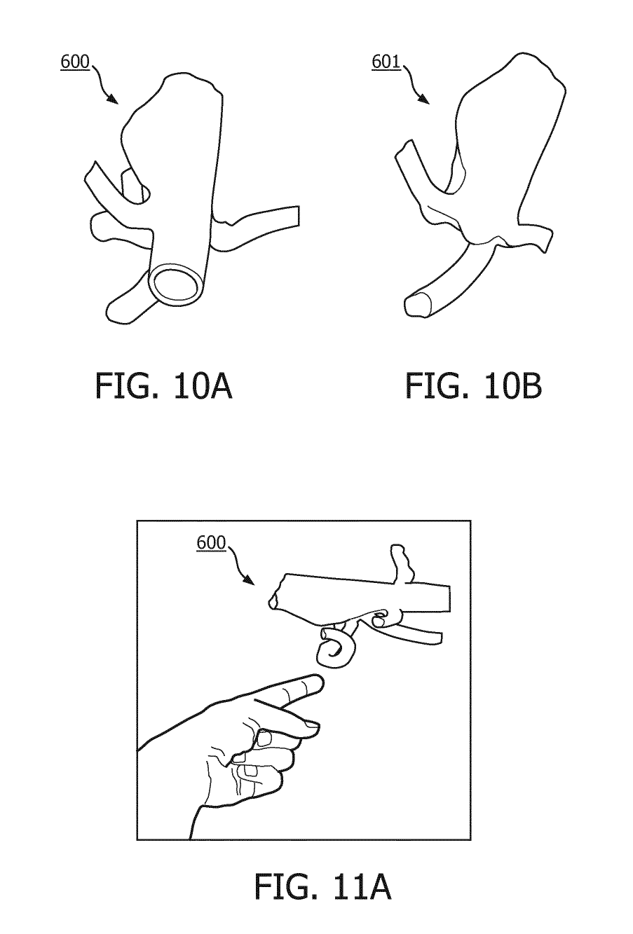

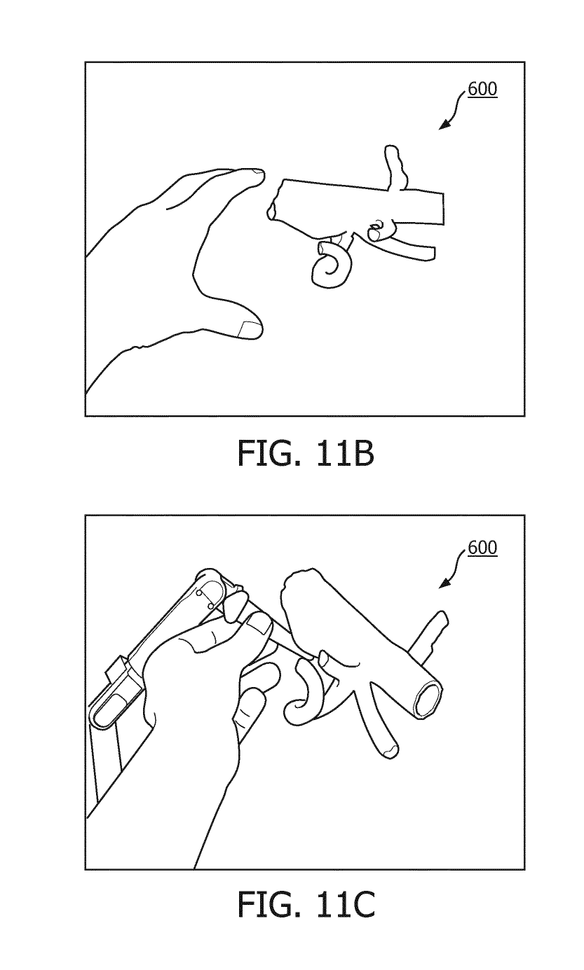

For clarity purposes in describing the anatomical model medical procedure of FIG. 9B, described embodiments for FIGS. 10A-11C of an anatomical model 40 as a hologram will be limited to abdominal aortic aneurysm and described embodiments of an imaging system 50 will be limited to a CT imaging system. Nonetheless, those having ordinary skill in the art of the present disclosure will appreciate the numerous and varied embodiments of an anatomical model and an imaging system applicable to anatomical model medical procedures of the present disclosure.

Referring to FIG. 9B, similar to anatomical model medical procedure 100 (FIG. 1B), an anatomical model medical procedure 100′ of the present disclosure implements an anatomical model acquisition phase 110′, a position planning phase 150′ and a tool guidance phase 190′ for conducting an imaging, a diagnosis and/or a treatment of a subject patient anatomy.

Anatomical model acquisition phase 110′ incorporates an image based hologram generation 400 as an addition to anatomical model acquisition phase 110 (FIG. 1B) as previously described herein.

In practice, anatomical model acquisition phase 110 involves a generation of by hologram control module 310 (FIG. 9A) of a holographic anatomical model 40 and/or a holographic tool replica 30 as previously described herein. Additionally, in practice, hologram control module 310 may apply anatomical model enhancements 140 as previously described herein to a holographic anatomical model 40 including, but not limited to:
1. a color changing of a portion or an entirety of the holographic anatomical model 40 to reflect a physical characteristic thereof (e.g., a patient's blood pressure, heart rate and other vital signs);
2. a changing of a size and/or location/orientation of the holographic anatomical model 40 to mimic any actual anatomical motion due to breathing, heat beat, etc.; and
3. a shading of the holographic anatomical model 40 to highlight a current imaging position of an interactive tool 301 with respect to the holographic anatomical model 40.

In practice, the holographic anatomical model 40 and/or a holographic tool replica 30 may be utilized during position planning phase 150′ and/or tool guidance phase 190′.

By example, a pre-operative CT scan of thoracic region of a patient by an imaging system 50 (FIG. 9A) is segmented by hologram control module 310 (FIG. 9A) whereby an augmented reality system 300 (FIG. 9A) is operated to generate a patient-specific holographic 3D model 600 of the AAA and branching vessels of an aorta. FIG. 10A illustrates a patient-specific holographic 3D model of such an AAA and branching vessels of an aorta generated by the use of Hololens™ commercially offered by Microsoft. As shown in FIG. 10B, an operator may use an interactive tool 301 (FIG. 1B) to slice through the model 600 to create model 601 for looking at various aspects of model 601 via a positioning of his or her head with respect to the model 601 to thereby ascertain a better understanding of the anatomy.

In practice, prior to or subsequent to any editing of model 600, an operator may interact with model 600 or model 601 in a variety of way for path planning purposes and/or tool guidance purposes including, but not limited to:
1. The operator pointing to 3D model 600 or 3D model 601 to add a ring landmarks for the ostea;
2. The operator utilizing a finger as a pointer to define a c-arm position for fluoroscopy acquisition, such as, for example, as shown in FIG. 11A;
3. The operator orienting the 3D model 600 or the 3D model 601 using gestures to a desired view, such as, for example, as shown in FIG. 11:
4. A utilization of a supplemental augment reality system (e.g., the Flexivision) whereby a 2D display of the supplement system may mimic that same orientation and position to thereby show pre-operative CT reconstruction;
5 The operator may position a 3D hologram of an endograft with respect to the 3D model 600 or the 3D model 610 to thereby practice positioning of an endograft; and
6. The operation may utilizes an encoded pointer (physical) to interact with the 3D model 600 or the 3D model 610 to define a landing zone for the endograft, such as, for example, as shown in FIG. 11C.

Referring back to FIG. 9B, position planning phase 150′ and tool guidance phase 190′ incorporates kinematics constraints as an addition to respective position planning phase 150 (FIG. 1B) and tool guidance phase 190 (FIG. 1B) as previously described herein.

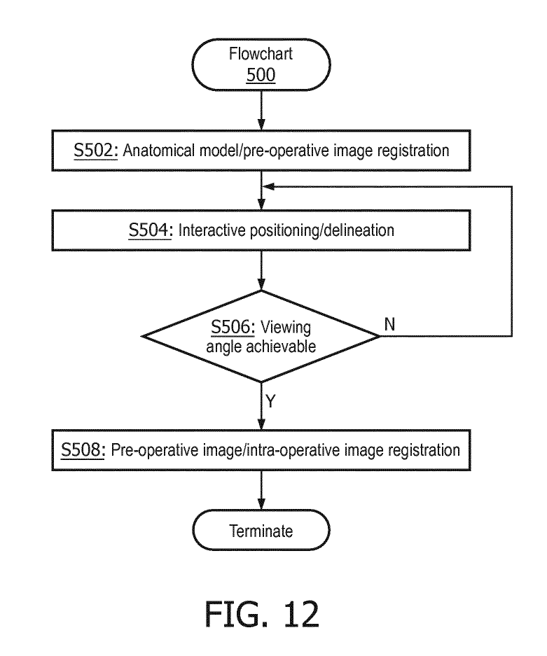

FIG. 12 illustrates a flowchart 500 representative of a kinematic control method of the present disclosure as incorporated in position planning phase 150′ and tool guidance phase 190′.

Referring to FIG. 12, a stage S502 of flowchart 500 encompasses a registration by path planning module 95 (FIG. 9B) or tool guidance module 96 (FIG. 9B) of a holographic anatomical model 40 to a pre-operative CT image of the patient anatomy via any suitable registration technique as known in the art of the present disclosure.

A stage S504 of flowchart 500 encompasses an interactive positioning of interactive tool 301 with respect to the holographic anatomical model to thereby delineate an imaging angle of interest. Stage S504 may further encompass a simulated viewing of the pre-operative CT image of the patient anatomy to facilitate the interactive positioning of interactive tool 301 with respect to the holographic anatomical model.

In one embodiment of stage S504, a tracked pointer, a hand gesture, or the like may be utilized to delineate the viewing angle of interest.

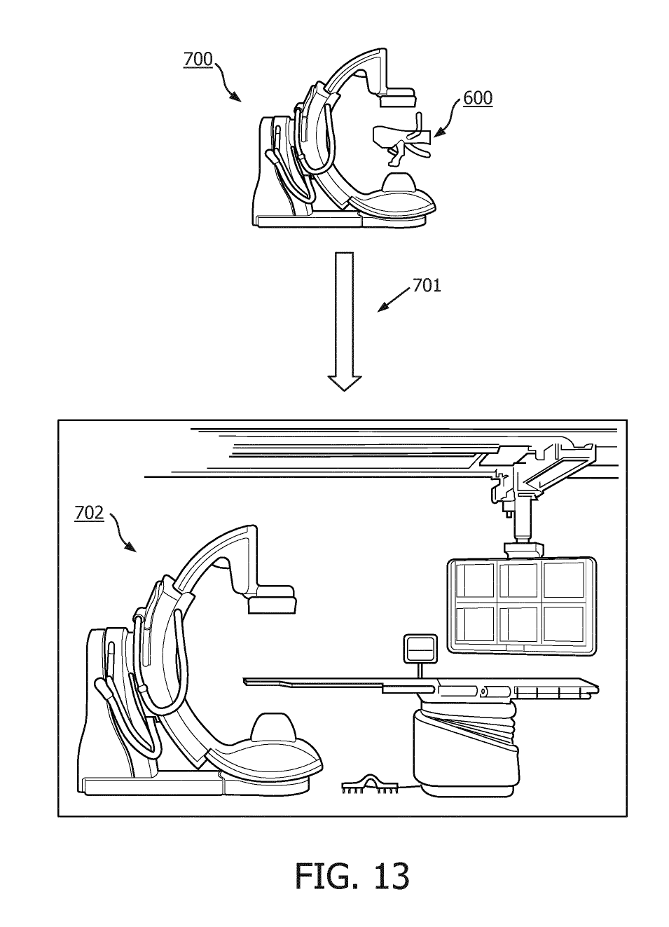

In a second embodiment of stage S504, an accurate kinematic scaled holographic anatomical model of imaging system 50 (FIG. 9B) may be positioned with respect to the holographic anatomical model to delineate the viewing angle of interest. For example, FIG. 13 illustrates an accurate kinematic scaled holographic anatomical model of a CT imaging system 700 positioned with respect to 3D model 600 to delineate the viewing angle of interest of the AAA.

Referring back to FIG. 12, a stage S506 encompasses a determination by path planning module 95 (FIG. 9B) or tool guidance module 96 (FIG. 9B) if the viewing angle is achievable as known in the art of the present disclosure based on any kinematic constraint(s) associated with an intra-operative imaging system.

If the viewing angle is not achievable, then the operator is notified via feedback as previously described herein whereby the operator may return to stage S504 to execute a new interactive positioning of interactive tool 301 with respect to the holographic anatomical model.

If the viewing angle is achievable, then path planning module 95 (FIG. 9B) or tool guidance module 96 (FIG. 9B) proceeds to a stage S508 of flowchart 500 to communicate, the viewing angle to the intra-operative imaging system for purposes of viewing the patient anatomy. For example, FIG. 13 illustrates a communication 701 of a viewing angle to an intra-operative CT imaging system 702 for purposes of positioning the C-arm gantry.

In practice, stages S502-S508 may be executed position planning phase 150′ (FIG. 9B), tool guidance phase 190′ (FIG. 9B) or a combination thereof.

Referring to FIGS. 1-13, those having ordinary skill in the art of the present disclosure will appreciate numerous benefits of the present disclosure including, but not limited to, an intuitive control of medical tools during an execution of a medical procedure.

Furthermore, as one having ordinary skill in the art will appreciate in view of the teachings provided herein, features, elements, components, etc. described in the present disclosure/specification and/or depicted in the Figures may be implemented in various combinations of electronic components/circuitry, hardware, executable software and executable firmware and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk read only memory (CD-ROM), compact disk read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive anatomical models for position planning and tool guidance during a medical procedure (which embodiments are intended to be illustrative and not limiting), it is noted that modifications and variations can be made by persons having ordinary skill in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. An anatomical model medical suite for executing an anatomical model medical procedure, the anatomical model medical suite comprising:
at least one of a medical tool and a tool replica,
wherein the medical tool is for conducting at least one of an imaging, a diagnosis and a treatment of the patient anatomy, and
wherein the tool replica is one of a physical representation or a virtual representation of the medical tool; and
a medical procedure controller,
wherein the medical procedure controller is structurally configured to control at least one of a position planning and a tool guidance of the medical tool relative to the patient anatomy derived from at least one of a position planning and a tool guidance of at least one of the medical tool relative to an anatomical model and a tool replica relative to the anatomical model, and
wherein the anatomical model is a physical representation of the patient anatomy.

2. The anatomical model medical suite of claim 1,
wherein the medical procedure controller is further structurally configured to control a generation of a model profile of the anatomical model derived from an imaging of the patient anatomy.

3. The anatomical model medical suite of claim 2,
wherein the medical procedure controller is further structurally configured to control an incorporation of at least one of physiologically-relevant information and procedural-relevant information into the model profile of the anatomical model.

4. The anatomical model medical suite of claim 2,
wherein the medical procedure controller is further structurally configured to control an incorporation of the position planning of at least one of the medical tool and the tool replica relative to the anatomical model into the model profile of the anatomical model.

5. The anatomical model medical suite of claim 1, wherein the medical procedure controller controlling a position planning of the medical tool relative to the patient anatomy includes:
the medical procedure controller being further structurally configured to control a procedural positioning of the medical tool relative to the patient anatomy derived from a procedural positioning of the at least one of the medical tool and the tool replica relative to the anatomical model.

6. The anatomical model medical suite of claim 1, wherein the medical procedure controller controlling a tool guidance of the medical tool relative to the patient anatomy includes:
the medical procedure controller being further structurally configured to control a procedural positioning of the medical tool relative to the patient anatomy derived from a procedural positioning of the tool replica relative to the anatomical model.

7. The anatomical model medical suite of claim 1, wherein the medical procedure controller controlling a tool guidance of the medical tool relative to the patient anatomy includes:
the medical procedure controller being further structurally configured to control a procedural positioning of the tool replica relative to the anatomical model derived from a procedural positioning of the medical tool relative to the patient anatomy.

8. The anatomical model medical suite of claim 1, further comprising:
a tracking system in communication with the medical procedure controller,
wherein the tracking system is structurally configured to generate tracking data informative of a positioning of the at least one of the medical tool and the tool replica relative to the anatomical model; and
wherein, responsive to a generation of the tracking data by the tracking system, the medical procedure controller controls the at least one of the position planning and the tool guidance of the medical tool relative to the patient anatomy derived from at least one of the position planning and the tool guidance of the at least one of the medical tool and a tool replica relative to the anatomical model.

9. The anatomical model medical suite of claim 1, further comprising:
a robotic system in communication with the medical procedure controller,
wherein the robotic system is structurally configured to generate pose data informative of a real-time pose of a tool robot relative to at least one of the patient anatomy and the anatomical model; and
wherein, responsive to a generation of the pose data by the robotic system, the medical procedure controller controls the at least one of the position planning and the tool guidance of the medical tool relative to the patient anatomy derived from at least one of the position planning and the tool guidance of the at least one of the medical tool and a tool replica relative to the anatomical model.

10. The anatomical model medical suite of claim 1, further comprising:
and an augmented reality system in communication with the medical procedure controller,
wherein the augmented reality system is structurally configured to control a user interaction with at least one hologram; and
wherein, responsive to the user interaction with the at least one hologram, the medical procedure controls the at least one of the position planning and the tool guidance of the medical tool relative to the patient anatomy derived from at least one of the position planning and the tool guidance of the at least one of the medical tool and a tool replica relative to the anatomical model.

11. The anatomical model medical suite of claim 1, further comprising:
the medical procedure controller controls at least one of the position planning and the tool guidance of the at least one of the medical tool and a tool replica relative to the anatomical model based on at least one kinematic constraint of the medical tool.

12. An anatomical model medical suite for executing an anatomical model medical procedure,
the anatomical medical procedure including a medical tool for conducting at least one of an imaging, a diagnosis and a treatment of the patient anatomy,
the anatomical model medical suite comprising:
at least one of an imaging system, a tracking system, a robotic system and an augmented reality system; and
a medical procedure controller,
wherein the medical procedure controller is structurally configured in communication with the at least one of the imaging system, the tracking system, the robotic system and the augmented reality system to control at least one of a position planning and a tool guidance of the medical tool relative to the patient anatomy derived from at least one of a position planning and a tool guidance of at least one of the medical tool relative to the anatomical model and a tool replica relative to the anatomical model, and
wherein the anatomical model is a physical representation of the patient anatomy, and
wherein the tool replica is one of a physical representation or a virtual representation of the medical tool.

13. The anatomical model medical suite of claim 12,
wherein the imaging system is structurally configured to generate imaging data ID illustrative of an imaging of the patient anatomy; and
wherein, responsive to a generation of the imaging data by the imaging system, the medical procedure controller is further structurally configured to control a generation of a model profile of the anatomical model derived from the imaging of the patient anatomy.

14. The anatomical model medical suite of claim 13,
wherein the medical procedure controller is further structurally configured to control an incorporation of at least one of physiologically-relevant information and procedural-relevant information into the model profile of the anatomical model.

15. The anatomical model medical suite of claim 13,
wherein the medical procedure controller is further structurally configured to control an incorporation of the position planning of at least one of the medical tool and the tool replica relative to the anatomical model into the model profile of the anatomical model.

16. The anatomical model medical suite of claim 12, further comprising:
wherein the tracking system is structurally configured to generate tracking data informative of a positioning of the at least one of the medical tool and the tool replica relative to the anatomical model; and
wherein, responsive to a generation of the tracking data by the tracking system, the medical procedure controller controls the at least one of the position planning and the tool guidance of the medical tool relative to the patient anatomy derived from at least one of the position planning and the tool guidance of the at least one of the medical tool and a tool replica relative to the anatomical model.

17. The anatomical model medical suite of claim 12,
wherein the robotic system is structurally configured to generate pose data informative of a real-time pose of a tool robot relative to at least one of the patient anatomy and the anatomical model; and
wherein, responsive to a generation of the pose data by the robotic system, the medical procedure controller controls the at least one of the position planning and the tool guidance of the medical tool relative to the patient anatomy derived from at least one of the position planning and the tool guidance of the at least one of the medical tool and a tool replica relative to the anatomical model.

18. The anatomical model medical suite of claim 12,
wherein the augmented reality system is structurally configured to control a user interaction with the anatomical model a user interaction with at least at least one hologram; and
wherein, responsive to the user interaction with the at least one hologram, the medical procedure controller controls the at least one of the position planning and the tool guidance of the medical tool relative to the patient anatomy derived from at least one of the position planning and the tool guidance of the at least one of the medical tool and a tool replica relative to the anatomical model.

19. The anatomical model medical suite of claim 12, further comprising:
the medical procedure controller controls at least one of the position planning and the tool guidance of the at least one of the medical tool and a tool replica relative to the anatomical model based on at least one kinematic constraint of the medical tool.

20. An anatomical model medical procedure, comprising:
providing an anatomical model as a physical representation of a patient anatomy;
providing at least one of a medical tool and a tool replica,
wherein the medical tool is for conducting at least one of an imaging, a diagnosis and a treatment of the patient anatomy; and
wherein the tool replica is one of physical representation or a virtual representation of the medical tool; and
controlling, by a medical procedure controller at least one of a position planning and a tool guidance of the medical tool relative to the patient anatomy as derived from at least one of a position planning and a tool guidance of at least one of the medical tool relative to the anatomical model and a tool replica relative to the anatomical model.

21. The anatomical model medical procedure of claim 20, further comprising:
controlling, by the medical procedure controller a generation of at least one of a manufacturing profile of the anatomical model derived from an imaging of the patient anatomy or an atlas profile of the anatomical model derived from the imaging of the patient anatomy.

22. The anatomical model medical procedure of claim 20, wherein controlling, by the medical procedure controller the position planning of the medical tool relative to the patient anatomy includes at least one of:
controlling, by the medical procedure controller a generation of pose commands for at least one a procedural positioning of the medical tool relative to the patient anatomy derived from a planned path of the at least one of the medical tool and the tool replica delineated within an imaging of the anatomical model; and
controlling, by the medical procedure controller a generation of pose commands for at least one of a procedural positioning of the medical tool relative to the patient anatomy derived from at least one planned positioning of the least one of the medical tool and the tool replica relative to the anatomical model.

23. The anatomical model medical procedure of claim 20, wherein controlling, by the medical procedure controller of the tool guidance of the medical tool relative to the patient anatomy includes at least one of:
controlling, by the medical procedure controller a generation of pose commands for at least one procedural positioning of the medical tool relative to the patient anatomy derived from procedural pose positions of the tool replica relative to the anatomical model; and
controlling, by the medical procedure controller a generation of pose commands for at least one procedural positioning of the tool replica relative to the anatomical model derived from procedural pose positions of the medical tool relative to the patient anatomy.

24. The anatomical model medical procedure of claim 20,
wherein, responsive to at least one of tracking data and pose data, the medical procedure controller controls the at least one of the position planning and the tool guidance of the medical tool relative to the patient anatomy derived from at least one of the position planning and the tool guidance of at least one of the medical tool and a tool replica relative to the anatomical model;
wherein the tracking data is informative of a positioning of the at least one of the medical tool and the tool replica relative to the anatomical model; and
wherein the pose data informative of a real-time pose of a tool robot relative to at least one of the patient anatomy and the anatomical model.

25. The anatomical model medical procedure of claim 20, further comprising:
user interacting with a hologram,
wherein, responsive to a user interaction with the at least one hologram, the medical procedure controller controls the at least one of the position planning and the tool guidance of the medical tool relative to the patient anatomy derived from at least one of the position planning and the tool guidance of the at least one of the medical tool and a tool replica relative to the anatomical model.

26. The anatomical model medical procedure of claim 20,
wherein the medical procedure controller controls at least one of the position planning and the tool guidance of the at least one of the medical tool and a tool replica relative to the anatomical model based on at least one kinematic constraint of the medical tool.
